(19) United States
(12) Patent Application Publication
(10) Pub. No.: US 2024/0304334 A1
(43) Pub. Date: Sep. 12, 2024

(54) CLOTTABILITY-BASED PERSONALIZED TREATMENT (CPT) SYSTEM

(71) Applicant: San PUN, Riehen (CH)

(72) Inventor: San PUN, Riehen (CH)

(21) Appl. No.: 18/572,143

(22) PCT Filed: Jun. 29, 2022
(86) PCT No.: PCT/EP2022/068005

§ 371 Date: Dec. 19, 2023

(30) Foreign Application Priority Data
Jun. 29, 2021 (EP) 21182542.7

(51) Int. Cl. G16H 50/30; A61K 45/06; G16H 10/40; G16H 10/60

(52) U.S. Cl. CPC G16H 50/30; A61K 45/06; G16H 10/40; G16H 10/60

(57) ABSTRACT

The present invention provides a method for modelling clottability of a blood sample, comprising determining clottability of blood samples or providing blood samples with known clottability due to the direct effects of biological activities of immune and blood coagulation systems on clottability; determining and attributing a health status and risk factor for thrombosis and/or bleeding to the donor having provided the respective sample; determining the pharmacodynamic effect(s) of one or more drug(s) including anti-inflammatory, anticoagulant(s) and the corresponding therapeutic range(s) on the blood sample to reduce inflammation and/or the risk(s) of thrombosis and/or bleeding; and modelling the clottability of a blood sample after administering the most effective therapeutic strategy involving drugs including anticoagulant to said blood sample.

### FIELD OF THE INVENTION

The present invention provides a method for modelling clottability of a blood sample, comprising determining clottability of blood samples or providing blood samples with known clottability due to the direct effects of biological activities of immune and blood coagulation systems on clottability; determining and attributing a health status and risk factor for thrombosis and/or bleeding to the donor having provided the respective sample; determining the pharmacodynamic effect(s) of drug(s) including anti-inflammatory, anticoagulant(s) and the corresponding therapeutic range(s) to reduce inflammation and/or the risk(s) of thrombosis and/or bleeding; and modelling the clottability of a blood sample after administering the most effective therapeutic strategy involving drugs including anticoagulant. The invention also provides a method for providing a personalized drug treatment regimen of a patient having pathological inflammation and/or increased risk(s) of thrombosis and/or bleeding due to the undesirable activities of immune and/or blood coagulation systems, the method comprising determining clottability of a blood sample of said patient or providing a known clottability value for said patient; and providing a personalized drug treatment regimen based on the model of clottability provided herein. This personal health status assessment and personalized treatment system utilizes clottability as one of the main parameters, without excluding the contributions from other parameters.

### BACKGROUND OF THE INVENTION

The pharmacological remedies for treatment and/or prevention of thrombosis are some of the most frequently prescribed medications worldwide. These pharmaceuticals are called anticoagulants and are blood thinners, which prevent and/or reduce coagulation of the blood by prolonging the clotting time. These drugs are used in the context with cardiovascular conditions, cancer, strokes, sepsis, and others.

Vitamin K antagonists, as one example, belong to the group of the most commonly prescribed anti-thrombotic drugs and are in use for more than 50 years (Zirlik and Bode 2016). Such drugs are for example fluindione, warfarin or coumarins. Dosage of these drugs is usually based on the PT/INR (prothrombin time/international normalized ratio) clotting assay. The PT/INR is determined in blood plasma of patients having received the drug for some time. The assay generalizes the dosage of the vitamin K antagonist and does not provide any output on the differences at individual level, in vivo biochemical and physiological properties or the precise dosage requirements.

Other types of anticoagulants are direct thrombin inhibitors (DTIs), which can be administered orally (e.g. dabigatran) or via intravenous infusion/injection (e.g. argatroban, hirudin). They can be small chemicals, larger peptides or peptide-like compounds targeting clotting factor thrombin with sufficient affinity and specificity. The inhibitory effects of DTIs are fluctuating daily, due to absorption, distribution, metabolism and excretion, such that the drug concentrations increase, reaching the peak shortly after drug intake. The drug concentrations in plasma decrease by drug elimination through the kidneys and other routes. Furthermore, orally administered direct FXa inhibitors (DXals), such as rivaroxaban, edoxaban, apixaban, heparin, or heparin-like drugs are small chemical compounds targeting clotting factor FXa. However, the inhibitory effects of vitamin K antagonists on delaying clotting are more durable or longer lasting than DTIs and DXals. Even if the extended clotting process in blood plasma was slowed down using a prescribed drug dosage, treatment is rather arbitrary, since thrombosis still occurs. Moreover, in case of excessive use of antithrombotic drugs, bleeding events increase.

Additionally, many patients are being treated not just with a single anticoagulant treatment. Most patients are being prescribed with many other drug types, such as anti-platelet, anti-inflammatory, drug metabolizer modulator, anti-high blood pressure, cellular transporter modulator, etc. All these drug-drug interactions are common and the net effects at each time point during the pharmacokinetics of the multi-drug treatment is a black box. For example, a combination of anti-platelet and anticoagulant treatment has an increased risk of bleeding, compared to single-drug treatment; on the other hand, the single-drug treatment poses an increased risk of thrombosis for certain individuals. As an example of a common anti-thrombotic treatment regimen, the risk of major bleeding increases at least 2-fold when anti-platelet/anti-inflammation (e.g. aspirin) is used in combination with an anticoagulant.

Hence, problems of currently used anticoagulant drug prescriptions for treatment and/or prevention of thrombosis are that the anticoagulant management is empirical and extrapolated from populational studies. Moreover, patients on the commonly prescribed regimens still suffer from life-threatening thrombosis or from bleedings of mild to life-threatening nature while under anticoagulant treatment.

Precision medicine is an approach for prevention, diagnosis, treatment, and monitoring of diseases that includes individual variability in biology, environment, and lifestyle of a patient. Patient biomarker data and diagnostic assays drive healthcare decision-making by helping physicians to identify the right treatment for patients and monitor their disease. Beyond that, biomarkers and accompanied diagnostics in precision medicine support the pipeline of drug companies by facilitating clinical trial design and execution, accelerating drug development, and informing the design of early pipeline choices. The key issue is the access to the diagnostics of a reliable biomarker.

Clottability biomarker is a novel biomarker which is able to indicate the status of both immune and blood coagulation systems, since both systems are tidely coupled to each other, e.g. activation of chronic inflammation due to autoimmunity can be observed in the activation of blood coagulation system. Anticoagulant may be exemplified as one of the main drug treatment types described in this patent, many diseases involving immune and blood coagulation systems are able to make use of such biomarker as a parameter, in combination of other parameters, to come up with a personalized health assessment, and hence a personalized treatment regimen with reduced side effects and increased treatment efficacy, such as bleeding and thrombosis in the case of single and/or combined anti-thrombotic treatment.

There is thus a need in the art to provide patient-specific treatment options to reduce the risk of thrombosis and/or bleeding, as in the example of anti-thrombotic treatment.

### SUMMARY OF THE INVENTION

The invention relates to a method for classifying the risk and/or status of a subject for inflammatory and/or clotting dysregulation events, the method comprising the steps of:
a) determining or retrieving input data, wherein the input data comprises
   i) a clottability biomarker, wherein the clottability biomarker is determined in a blood sample of a subject by at least two different assays; and
   ii) patient background information;
b) comparing the input data to a risk and/or status reference pattern, wherein the risk and/or status reference pattern is obtained from at least two reference subjects wherein at least one of the reference subjects has previously had an inflammatory and/or clotting dysregulation event; and
c) classifying the risk and/or status of the subject for inflammatory and/or clotting dysregulation events based on the comparison obtained in (b).

The inventors have surprisingly found that the use of a novel biomarker, clottability, and the measurement of this biomarker to track the coagulation or thrombin activity before and after anticoagulant treatment provides the possibility to offer a patient-specific anticoagulant and anti-bleeding treatment of reducing the risk of thrombosis and/or bleeding. As already indicated in the previous section, this biomarker which is an indicator of how active the systems of immune and coagulation during disease, will respond to pharmaceutical treatments having effects on either or both systems. This patent mainly exemplifies the use of anticoagulant and its pharmacodynamic effects on this biomarker. The current invention thus provides a personalized computational solution based on a novel biomarker by prescribing personalized and precise dosage of an anticoagulant drug, and thus improving the medication regimen. Moreover, it was surprisingly found that the measurement of the clottability can be easily applied to guide the personalized treatment of a patient, since this diagnostic test can be adopted by automated hematology instruments.

In a further embodiment, the invention relates to a method for providing a personalized anticoagulant and anti-bleeding treatment regimen of a patient, the method comprising:
(a) determining clottability of a blood sample of said patient or providing a known clottability value for said patient,
(b) providing a personalized anticoagulant and anti-bleeding treatment regimen based on the model of clottability obtained by the method of the invention.

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. In the claims the word “comprising” does not exclude other elements or steps, and the indefinite article “a” or “an” does not exclude a plurality. A single unit may fulfill the functions of several features recited in the claims. Any reference signs in the claims should not be construed as limiting the scope. As used herein, “and/or” should be understood to mean either one, or both alternatives.

Unless defined otherwise, all technical and scientific terms used herein have the same meaning as is commonly understood by one skilled in the art to which the subject matter herein belongs. As used herein, the following definitions are supplied herein for the purpose of describing particular embodiments only and is not intended to limit the scope of the present invention.

Accordingly, the invention relates to, inter alia, the following embodiments:

1. A method for classifying the risk and/or status of a subject for inflammatory and/or clotting dysregulation events, the method comprising the steps of:
a) determining or retrieving input data, wherein the input data comprises
   i) a clottability biomarker, wherein the clottability biomarker is determined in a blood sample of a subject by at least two different assays; and
   ii) patient background information;
b) comparing the input data to a risk and/or status reference pattern, wherein the risk and/or status reference pattern is obtained from at least two reference subjects wherein at least one of the reference subjects has previously had an inflammatory and/or clotting dysregulation event; and
c) classifying the risk and/or status of the subject for inflammatory and/or clotting dysregulation events based on the comparison obtained in (b).

2. The method of embodiment 1, wherein the assays are a combination of a clot-based fibrinogen test and an enzyme-based fibrinogen test.

3. The method of embodiment 2, wherein the enzyme-based fibrinogen test is a clot-independent enzyme-based fibrinogen test.

4. The method of embodiment 2, wherein the clot-based fibrinogen test is selected from determination of the prothrombin time (PT), determination of partial thromboplastin time (PTT), and Clauss-test.

5. The method of any one of embodiments 2 to 4, wherein the enzyme-based fibrinogen test involves catalytic cleavage by a serine endopeptidase.

6. The method of any one of embodiments 2 to 5, wherein the enzyme-based fibrinogen test involves catalytic cleavage of fibrinogen by snake venom serine endopeptidase, preferably by venombin A.

7. The method of any one of embodiments 2 to 6, comprising measuring the proteolytic activity of a serine endopeptidase which is inversely proportional to the fibrinogen level in said sample.

8. The method of any one of embodiments 1 to 7, wherein the patient background information comprises or consists of body weight, sex, age and kidney function.

9. The method of any one of embodiments 1 to 8, wherein the risk and/or status reference pattern is a machine learning model obtained by training on a dataset of reference subjects and wherein comparing the input data to a risk and/or status reference pattern comprises inputting the input data in the machine learning model.

10. The method of any one of embodiments 1 to 9, wherein classifying the risk and/or status of the subject for inflammatory and/or clotting dysregulation event is classifying the risk and/or status of a subject for thrombosis and/or bleeding.

11. A method for prediction of the risk and/or status of a subject for inflammatory and/or clotting dysregulation events, the method comprising the steps of:
a) determining a risk and/or status progression indicator by the steps of:
   i) classifying the risk and/or status of a subject for inflammatory and/or clotting dysregulation events according to the method of any one of the embodiments 1 to 10 during at a first time point; and
   ii) determining or retrieving a clottability biomarker of a blood sample of the subject at a second timepoint, wherein the clottability biomarker is determined in the blood sample of the subject by at least two different assays; and
b) comparing the risk and/or status progression indicator to a prediction reference pattern, wherein the prediction reference pattern is obtained from at least two reference subjects wherein at least one of the reference subjects has previously had an inflammatory and/or clotting dysregulation event and wherein the risk and/or status progression(s) of the reference subject(s) is/are known;
c) predicting the risk and/or status of a subject based on the comparison obtained in (b).

12. The method of embodiment 11, wherein the prediction reference pattern is a machine learning model obtained by training on a dataset of reference subjects and wherein comparing the risk and/or status progression indicator to a prediction reference pattern comprises inputting the input data in the machine learning model.

13. A method for monitoring treatment response of a subject for inflammatory and/or clotting dysregulation events during treatment, the method comprising the steps of:
a) determining a treatment progression indicator by the steps of:
   i) classifying the risk and/or status of a subject for inflammatory and/or clotting dysregulation events according to the method of any one of the embodiments 1 to 10 at a first timepoint;
   ii) determining or retrieving a clottability biomarker of a blood sample of the subject on at least one second timepoint, wherein the clottability biomarker is determined in the blood sample of the subject by at least two different assays; and
   iii) 1.) an administration timepoint of the anti-inflammatory, anti-platelet, anticoagulation and/or pro-coagulation and/or anticoagulation compound, wherein the administration timepoint is between the first time point and the second timepoint; preferably the administration timepoint and an administered amount of the anti-inflammatory, anti-platelet, anticoagulation and/or pro-coagulation and/or anticoagulation compound; and
      2.) a pharmacodynamic response, wherein the pharmacodynamic response is calculated at least based on the clottability biomarker on the first timepoint, the clottability biomarker on the second time point and the administration timepoint, wherein the pharmacodynamic response is calculated at least based on the clottability biomarker on the first timepoint, the clottability biomarker on the second time point, the administration timepoint and the administered amount;
b) comparing the treatment progression indicator to a treatment response reference pattern, wherein the prediction reference pattern is obtained from at least two reference subjects wherein at least one of the reference subjects previously underwent treatment with an anti-inflammatory, anti-platelet, anticoagulation and/or pro-coagulation compound and wherein the treatment response(s) of the reference subject(s) is/are known;
c) monitoring treatment response of a subject based on the comparison obtained in (b).

14. The method of embodiment 13, wherein the treatment response reference pattern is a machine learning model obtained by training on a dataset of reference subjects and wherein comparing the treatment progression indicator to a treatment response reference pattern comprises inputting the treatment progression indicator in the machine learning model.

15. An anti-inflammatory, anti-platelet, anticoagulation and/or pro-coagulation compound for use in the treatment of a subject classified as being at risk and/or status for inflammatory and/or clotting dysregulation events according to the method of any one of the embodiments 1 to 10 and/or predicted to develop risk and/or status for inflammatory and/or clotting dysregulation events according to the method of embodiment 11 or 12.

16. A method of treatment for reducing the risk and/or improving the status of an inflammatory and/or clotting dysregulation event in a subject in need, the method comprising the steps of:
a) administering a therapeutically effective amount of a first anti-inflammatory, anti-platelet, anticoagulation and/or pro-coagulation compound; during a monitoring of the treatment response according to the method of any one of the embodiments 13 to 14 to a subject in need; and
b) administering a second anti-inflammatory, anti-platelet, anticoagulation and/or pro-coagulation compound to the subject in need if the treatment response to the first anti-inflammatory, anti-platelet, anticoagulation and/or pro-coagulation compound is insufficient according to the method of any one of the embodiments 13 to 14 and proceeding therapy with the first anti-inflammatory, anti-platelet, anticoagulation and/or pro-coagulation compound if the treatment response to the first anti-inflammatory, anti-platelet, anticoagulation and/or pro-coagulation compound is sufficient according to the method of any one of the embodiments 13 to 14 for reducing the risk and/or improving the status of an inflammatory and/or clotting dysregulation event in the subject in need.

17. The method of any one of embodiments 13 to 14, the compound for use of embodiment 15 or the method of treatment of embodiment 16, wherein the compound is a compound selected from the group consisting of Vitamin K antagonists, in particular fluindione, warfarin or coumarins, direct thrombin inhibitors, in particular dabigatran, argatroban or hirudin, and direct FXa inhibitors, in particular rivaroxaban, edoxaban, apixaban, heparin, or heparin-like drugs.

18. The method of any one of embodiments 13 to 14, the compound for use of embodiment 15 or the method of treatment of embodiment 16, wherein the compound is a compound selected from the group consisting of: non-steroidal anti-inflammatory drugs, corticosteroids, rapamycin, high density lipoproteins, HDL-cholesterol elevating compounds, rho-kinase inhibitors, anti-malarial agents, acetaminophen, glucocorticoids, steroids, beta-agonists, anticholinergic agents, xanthine derivatives, sulphasalazine, penicillamine, anti-angiogenic agents, dapsone, psoralens, anti TNF agents, anti-IL-1 agents and statins.

19. The method of any one of embodiments 13 to 14, the compound for use of embodiment 15 or the method of treatment of embodiment 16, wherein the compound is a compound selected from the group consisting of: irreversible cyclooxygenase inhibitors, adenosine diphosphate (ADP) receptor inhibitors, phosphodiesterase inhibitors, protease-activated receptor-1 (PAR-1) antagonists, glycoprotein IIB/IIIA inhibitors, adenosine reuptake inhibitors, dipyridamole, thromboxane inhibitors and thromboxane receptor antagonists.

20. The method of any one of embodiments 13 to 14, the compound for use of embodiment 15 or the method of treatment of embodiment 16, wherein the compound is a coagulation factor that promotes clotting and/or reduces bleeding, preferably a compound selected from the group consisting of FVIII concentrate, Alphanate, Humate-P, NovoSeven, Eloctate, Feiba, prothrombin complex, Hemlibra, and tranexamic acid.

21. A storage device comprising computer-readable program instructions to execute the method according to any one of the embodiments 1 to 14, 17 to 20.

22. A server comprising the storage device of embodiment 21, at least one processing device for executing the computer-readable program instructions, and a network connection for receiving the input data.

23. A system for classification, prediction and/or monitoring a treatment response, the system comprising:
a) a measurement setup comprising a container for receiving a blood sample and reagents for determining a clottability biomarker, wherein the clottability biomarker is determined in the blood sample by at least two different assays;
b) a processing device for executing the computer-readable program instructions comprising the storage device of embodiment 21 and/or a network connection to a server, wherein the server is a server according to embodiment 22; and
c) an input and/or retrieval possibility, wherein the input and/or retrieval possibility enables the server and/or the processing device access to the patient background information.

Accordingly, in one embodiment, the invention relates to a method for classifying the risk and/or status of a subject for inflammatory and/or clotting dysregulation events, the method comprising the steps of: a) determining or retrieving input data, wherein the input data comprises: i) a clottability biomarker, wherein the clottability biomarker is determined in a blood sample of a subject by at least two different assays; and ii) patient background information; b) comparing the input data to a risk and/or status reference pattern, wherein the risk and/or status reference pattern is obtained from at least two reference subjects wherein at least one of the reference subjects has previously had an inflammatory and/or clotting dysregulation event; and c) classifying the risk and/or status of the subject for inflammatory and/or clotting dysregulation events based on the comparison obtained in (b).

The term “risk and/or status of a subject for inflammatory and/or clotting dysregulation events”, as used herein, refers to any measure or category that is indicative of events that occur upon dysregulation of the immune-, platelet- and/or coagulation system. In some embodiments, the risk and/or status of a subject for inflammatory and/or clotting dysregulation events described herein is a category or measure indicative of the risk and/or status of at least one selected from the group consisting of: acute inflammation, chronic low-grade inflammation, cardiovascular event and bleeding. In some embodiments, the risk and/or status of a subject for inflammatory and/or clotting dysregulation events described herein is a category or measure indicative of the risk and/or status of at least one selected from the group consisting of: thrombosis, stroke, angina, myocardial infarction bleeding. In some embodiments the thrombosis described herein is thromboembolic disease or venous thrombosis.

The term “clottability biomarker”, as used herein, refers to a biomarker determined by at least two different assays and indicative of clottability. In some embodiments, at least one of the assays to determine the clottability biomarker described herein is an enzyme-based fibrinogen test, preferably and enzyme-based fibrinogen test involves catalytic cleavage by a serine endopeptidase and/or catalytic cleavage of fibrinogen by snake venom serine endopeptidase, preferably by venombin A.

The term “patient background information”, as used herein, refers to any patient information that is not the clottability biomarker. In some embodiments the patient background information is at least one, at least two, at least three or at least four selected from the group consisting of disease history, vaccination status, liver function, height, BMI, infection status, treatment history, genetic type, metabolizer type, current treatment, body weight, sex, age, blood pressure and kidney function. In some embodiments the patient background information is sex, age and kidney function. The term “health status” as used herein may also be understood as patient background information.

The term “reference pattern”, as used herein, refers to a reference that is useful for classification of the input data that is obtainable from reference subjects. As such the reference pattern can be at least on threshold, a classification function, a model or a set of weights. In some embodiments, the reference pattern described herein is a trained machine learning model.

The term “reference subjects”, as used herein, refers to a plurality of subjects, for which a parameter for which they serve as a reference is known. In some embodiments, the reference subjects described herein comprise healthy and diseased subjects. In some embodiments, the reference subjects described herein consist of diseased subjects. In some embodiments, the reference subjects described herein are part of a clinical study such as a population study.

The inventors found that the clottability biomarker in combination with patient background information provides an efficient way to classify status and/or risk in subjects and can improve and personalize diagnosis, monitoring and/or treatment.

In certain embodiments, the present invention relates to a method for modelling clottability of a blood sample, the method comprising the steps of determining clottability of a blood sample or providing a blood sample with known clottability, wherein the clottability is determined by and/or provided from at least two different assays; determining and attributing a health status and risk factor for thrombosis and/or bleeding to the donor having provided the respective blood sample; determining a pharmacodynamic effect of one or more drug(s) and/or multiple-drug administrations, wherein the drug(s) and/or multiple-drug administrations comprise or consist of anti-inflammation, anti-platelet, and/or anticoagulation treatment; and the corresponding therapeutic range on the blood sample to reduce the risk; and modelling the clottability of a blood sample after administering the most effective drug to said blood sample.

As used herein, the term “thrombosis” refers to the formation of a blood clot inside a blood vessel, obstructing the flow of blood. Thrombosis may occur in veins (venous thrombosis) and arteries (arterial thrombosis). The term “bleeding” as used herein refers to extravasation of blood from the vessels of the circulatory system. Bleeding is usually stopped after a given time by blood clotting. However, as used herein “bleeding” relates to excessive bleeding due to impaired clot formation, in particular due to reduced fibrinogen concentration and/or excessive presence of factors inhibiting clotting.

As used herein, the term “clottability” relates to a value, quantitative (numeric) or qualitative, indicating the ability of blood to clot, preferably within a pre-determined time, preferably under specific conditions.

Blood clots are formed by fibrin, the activated and polymerized form of fibrinogen, together with platelets. Activation occurs through the protease thrombin that forms the fibrous, non-globular protein fibrin from fibrinogen. The activity of the coagulation pathway can be indicated by the activity of thrombin being activated via intrinsic and extrinsic pathways, with the participation of non-cellular and cellular components in the blood. This thrombin activity leaves its marking by converting fibrinogen into fibrin and fibrin-derived molecules, depending on the conditions. During this process, small soluble and insoluble polymers of various sizes and complexity are formed. These complex and various fibrin-derived molecules or complexes are indications of thrombin generation in vivo, and their presence in the blood increases the clottability or coagulability of the blood, hence they are called pro-coagulable factors. Current detection methods of such pro-coagulable factors, including the soluble fibrin, are difficult to perform and do not result in satisfactory results. Plasma with higher concentration of such pro-coagulable factors is able to clot at higher rate, compared with the same plasma without such pro-coagulable factors. Therefore, a plasma sample taken from a subject with negligible in vivo thrombin activation or generation contains negligible pro-coagulable factors, which is an ideal condition that rarely the case. When the plasma is taken from the same subject experiencing acute inflammation (e.g. endotoxin intoxication) a few hours before sampling, a portion of the total fibrinogen is converted to these pro-coagulable factors and the clottability of the plasma increases.

Once formed, these molecules and complexes are further processed by a protein degradation process termed fibrinolysis. Due to the fibrinolysis, these fibrin-derivatives are further processed to generate degradation products of various completeness called fibrin-degradation products (FDPs). These FDPs exhibit differential effects on the fibrin-polymerization or clot-formation efficiency. From early to late phases of fibrinolysis, these FDPs produce from highly to very little inhibitory effects on clotting, respectively. D-dimers of various forms are the products of such late phase fibrinolysis. These factors that inhibit fibrin polymerization are called anti-coagulable factors; their presence in the clotting reaction can slow down the clotting rate. The fibrinolysis activity is more pronounced when thrombin activity is reduced.

The term “clottability” as used herein is a measurement of these pro- and anti-coagulable factors in the plasma. It is based on one or more in vitro test(s) that is/are able to reflect the in vivo presence of these factors due to the recent or fresh activity of thrombin on fibrin(ogen). The skilled person is well-aware of this term representing both fibrins and fibrinogens of any forms and sizes and modifications. The general influences of these factors in clot-based assays (e.g. Clauss or other CCTs) are different degrees of acceleration (hyper-clottability) or deceleration (hypo-clottability).

The skilled person is aware that blood clots naturally occur at a wound site to prevent leakage. However, the skilled person is also aware that clotting may occur in situations where there is no physiological need thereof, potentially resulting in a reduced flowability of blood and thus thrombosis. Clotting may, however, also be reduced or even absent, resulting in continuous bleeding in case of rupture of a blood vessel. As such, the skilled person is aware that the physiologically important process of blood clotting may be positively or negatively altered in patients.

As used herein, the term “modelling clottability” refers to a process where the clottability, as defined above, is calculated/estimated for a blood sample for which the clottability has not been determined by use of one or more biochemical assay(s) as defined below. As such, the modelled clottability can be used to predict the expected clottability of a blood sample, for example upon treatment with an anticoagulant. It can thus be used to determine the optimal therapy for a patient in need of anticoagulant treatment in order to reduce the risk of the above defined effects of thrombosis and bleeding in case clots occur due to a reduced or increased clottability vis-á-vis a state where clotting ability is physiologically acceptable.

In a first step, the method of the invention comprises the step of determining clottability of blood samples or providing samples with known clottability. The clottability, as defined above, can be determined using blood samples or based on samples for which the clottability is known. The “blood samples” as used herein, may be obtained from a mammal, such as a human, but may also be obtained from a horse, cow, sheep, pig, primate, dog or mouse.

It is well established, that the clottability and its cardiovascular consequences such as bleeding and thrombosis are involving inflammation and coagulation (which includes platelet) systems. Accordingly, the influence of drugs affecting anti-inflammatory, anticoagulation and antiplatelet compounds can be modelled as described herein.

The term “anti-inflammation treatment” as used herein refers to a refers therapeutic agent for the treatment of an inflammatory disease or the symptoms associated therewith. In some embodiments, the anti-inflammatory compound described herein is at least one compound selected from the group consisting of: non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin, high density lipoproteins (HDL) and HDL-cholesterol elevating compounds, rho-kinase inhibitors, anti-malarial agents (e.g., hydroxychloroquine and chloroquine), acetaminophen, glucocorticoids, steroids, beta-agonists, anticholinergic agents, xanthine derivatives (e.g., methyl xanthines), sulphasalazine, penicillamine, anti-angiogenic agents, dapsone, psoralens, anti TNF agents, anti-IL-1 agents and statins, preferably infliximab, adalimumab, certolizumab pegol, golimumab, etanercept, curcumin, IL-1 RA, canakinumab, allopurinol, colchicine, pentoxifylline and oxypurinol. In certain embodiments, the anti-inflammation treatment is a therapeutic agent for the treatment of an inflammatory disease or the symptoms associated therewith, wherein the anti-inflammatory effect is achieved by inhibiting and/or reducing platelet's function.

The term “anti-platelet treatment” as used herein refers to a therapeutic agent for that reduces or inhibits platelet aggregation. In some embodiments, the anti-platelet treatment described herein is at least one compound selected from the group consisting of: irreversible cyclooxygenase inhibitors, adenosine diphosphate (ADP) receptor inhibitors, phosphodiesterase inhibitors, protease-activated receptor-1 (PAR-1) antagonists, glycoprotein IIB/IIIA inhibitors, adenosine reuptake inhibitors, dipyridamole, thromboxane inhibitors and thromboxane receptor antagonists. In some embodiments, the anti-platelet treatment described herein is at least one compound selected from the group consisting of: terutroban, vorapaxar, cilostazol, aspirin, triflusal, cangrelor, clopidogrel, prasugrel, ticagrelor, ticlopidine, abciximab, eptifibatide and tirofiban.

The term “anticoagulation treatment” as used herein refers to a drug comprising or consisting of any anticoagulation compound. In some embodiments, the anti-inflammatory compound described herein is at least one compound selected from the group consisting of: Vitamin K antagonists, coumarins, direct thrombin inhibitors, direct FXa inhibitors, heparin and heparin-like drugs. In some embodiments, the anti-inflammatory compound described herein is at least one compound selected from the group consisting of: fluindione, warfarin, dabigatran, argatroban, hirudin, rivaroxaban, edoxaban and apixaban.

The terms “pharmacodynamic effect” or “pharmacodynamic response” as described herein, refer to a measurable effect of drug treatment on the coagulation system or immune and coagulation systems and the physiological consequences of such effects. Examples of such drug-induced effects can be found in Examples 5, 6, 7 and 8. In some embodiments, the “pharmacodynamic effect” or “pharmacodynamic response” is derived from the clottability biomarker or changes in the clottability biomarker described herein.

In certain embodiments, the present invention relates to a method for modelling clottability of a blood sample, the method comprising the steps of determining clottability of blood samples or providing blood samples with known clottability; determining and attributing a health status and risk factor for thrombosis and/or bleeding to the donor having provided the respective sample; determining the pharmacodynamic effect of one or more anticoagulant(s) and the corresponding therapeutic range on the blood sample to reduce the risk; and modelling the clottability of a blood sample after administering the most effective anticoagulant to said blood sample.

One way to determine clottability is based on fibrinogen concentration in a blood sample. The determined fibrinogen concentration can be related to known fibrinogen concentrations to determine whether the blood sample will be normal-clottable, hyperclottable or hypoclottable. Within the present invention, one way to “determine clottability” involves using at least two assays to determine fibrinogen concentration and determining the difference between the at least two assay results. In this regard, it has been surprisingly found by the inventor that the clottability resulting from the above can serve as an indicator for the health status assessment and risk of the donor to suffer from thrombosis or bleeding, depending on a hyper- or hypoclottable state, respectively. In this regard, it was surprisingly found that for healthy donors, the resulting clottability as the difference of fibrinogen concentrations determined by at least two assays, preferably at different time points, may be between ≥−0.5 and ≤0.5. As such in general population, it was found that if two or more tests result in similar fibrinogen concentrations, i.e. a low variance, there is a low risk of developing thrombosis or bleeding and thus the donors are healthy with reduced activation of the immune and coagulation systems. On the other hand, if results from the at least two assays differ to an extent that the difference of determined fibrinogen concentrations is maintained much >0.5, the risk of developing thrombosis increases, as shown in the appended examples and FIG. 1. The risk of bleeding development increases when the clottability remains below the safe interval.

Clottability values may change, and thus are dynamic due to physiological processes and/or medication. Thus, clottability should be monitored regularly. The present invention provides a method for modelling the clottability before treatment and, as such, may reduce the empirical monitoring of clottability before and after treatment. At the same time, the methods of the invention may lead to a more rapid and accurate treatment decision and may thus reduce the risk of thrombosis and/or bleeding.

The method of the invention further comprises the step of determining and attributing a health status to the donor based on clottability modelling which reflects the activities or activations of the immune system and blood coagulation system. It is known that chronic activation of the immune system is associated with diseases such as cancer and other non-communicable diseases, and infections such as HIV infection, etc.

Further the method of the invention comprises the step of determining the pharmacodynamic effect of one or more anticoagulant(s) and the corresponding therapeutic range to reduce the risk for thrombosis and/or bleeding. In order to reduce the risk of thrombosis and/or bleeding, the pharmacodynamic effect of one or more anticoagulant(s) is determined. Exemplary, reducing thrombin activation and thus also clottability, for example from a hyperclottable to a normal-clottable state, minimizes the risk of thrombosis. An example of an increased risk for bleeding, in this context, is an overdose of an anticoagulant, and to some extent the excessive production of anti-coagulable factors.

Within the present invention preferred anticoagulant(s) may be selected from the group consisting of Vitamin K antagonists, in particular fluindione, warfarin or other coumarins, direct thrombin inhibitors, in particular dabigatran, argatroban or hirudin, and direct FXa inhibitors, in particular rivaroxaban, edoxaban, apixaban, heparin, or heparin-like drugs. Anticoagulants are drugs used for reducing the risk of blood clots, in particular in order to prevent thrombosis, pulmonary embolism, strokes in individuals having atrial fibrillation, valvular heart disease and/or artificial heart valves. Anticoagulants may be administered, for example, orally, intravenously, subcutaneously, parenterally, intra-arterially or topically. Preferably, anticoagulants may be administered orally or intravenously. There are different types of anticoagulants acting on different parts of the physiological processes leading to blood clot formation. In particular, “vitamin K antagonists” are natural or synthetic compounds, analogues or derivatives thereof, which inhibit the enzyme vitamin K epoxide reductase and thus inhibit the regeneration of vitamin K, which is an important co-factor in the blood clotting cascade. Further, “direct thrombin inhibitors” act as anticoagulants by directly inhibiting the enzyme thrombin, which is responsible for blood clotting. For example, dabigatran inhibits thrombin in the common coagulation pathway preventing fibrin formation from fibrinogen. “Direct FXa inhibitors” directly bind to the factor Xa, inhibiting its action in blood clotting. However, the type of anticoagulant used in the present invention is not particularly limited.

The method of the invention further encompasses the step of modelling the clottability after administering the most effective anticoagulant. The term “after administering”, as used herein, refers to the theoretical effect of the anticoagulant determined to be the most effective when administered to the patient. Within the present invention, “most effective” refers to the anticoagulant or combination of anticoagulants determined to result in the most advantageous physiological effect. The most advantageous physiological effect may be with respect to the reduction or increase of clottability but may also include factors such as safety and/or multiple drug use. Exemplary, a patient may no longer be at a hyperclottable state, if clottability is being reduced, or a patient may no longer be at a hypoclottable state, if clottability is being increased, after administration of the most effective anticoagulant.

In a further embodiment, the invention relates to the method of the invention, wherein determining clottability comprises a combination of a clot-based fibrinogen test and an enzyme-based fibrinogen test.

In certain embodiments, the invention relates to the method of the invention, wherein the assays are a combination of a clot-based fibrinogen test and an enzyme-based fibrinogen test.

Examples of both tests are disclosed in the publication WO2019/068940. The term “clot-based fibrinogen test”, as used herein, refers to the specific determination of fibrinogen activity, based on the time it takes for clot formation by evaluating the clotting process in which fibrinogen is converted into fibrin.

Within the present invention the term “clot-based fibrinogen test” includes all tests that relate to clotting reactions that are able to result in an interpolation regarding fibrinogen concentration. Examples of a clot-based fibrinogen test as used herein include the prothrombin time test (PT), the partial thromboplastin time test (PTT), and the Clauss-test (CCT). The term, “enzyme-based fibrinogen test”, as used herein, refers to a test determining fibrinogen concentration in blood based on competitive kinetic data by utilizing an enzyme and an artificial substrate. The “enzyme” may be selected from peptidase, protease, lipase, pectinase, amylase, or isomerase. Preferably, the enzyme used in the enzyme-based fibrinogen test of the invention is a protease. Example of an enzyme-based fibrinogen test is the true fibrinogen test (TFT) utilizing a protease, which selectively cleaves fibrinogen.

In this regard, the invention is at least in part based on the finding that in order to determine the clottability of a blood sample, a combined use of an assay comprising a clot-based fibrinogen test and an enzyme-based fibrinogen test can be the basis for effectively determining clottability. Exemplary, FIG. 3 illustrates the relationship between plasma fibrinogen concentrations obtained from a clot-based fibrinogen test and an enzyme-based fibrinogen test at different time points and the derived clottability in a healthy individual with acute inflammation.

Within the present invention the enzyme-based fibrinogen test may be a clot-independent enzyme-based fibrinogen test.

In certain embodiments, the invention relates to the method of the invention, wherein the enzyme-based fibrinogen test is a clot-independent enzyme-based fibrinogen test.

The term “clot-independent” refers to an enzyme-based fibrinogen test, which does not detect pro- and/or anti-coagulable factors. The term “pro-coagulable factors”, as used herein, refers to substances within the coagulation cascade which enhance the clotting efficiency. In particular, pro-coagulable factors are mostly soluble fibrin derivatives or thrombin-generated intermediates from fibrinogen, as well as some fibrin degradation products (FDPs) generated by fibrinolysis at an early stage. “Anti-coagulable factors” refer to substances within the coagulation cascade having the opposite effect by reducing the efficiency of clot formation. In particular, anti-coagulable factors are mostly FDPs of a later stage.

Within the present invention it is further preferred that the clot-based fibrinogen test is selected from determination of the prothrombin time (PT), determination of partial thromboplastin time (PTT), and Clauss-test.

In particular, a PT test indirectly measures the fibrinogen derived from the prothrombin time in seconds. Calibration is performed by calculating the prothrombin time on plasma containing a series of known fibrinogen concentration standards and plotting the optical change against the fibrinogen values. The optical change is converted to a fibrinogen value (Mackie et al. 2003).

PT is often used in combination with an aPTT (activated partial thromboplastin time) test, which can evaluate the amount and function of coagulation factors. The terms “partial thromboplastin time” or “PTT” refer to a blood test that determines the time it takes for blood to clot. The tests are used to diagnose unexplained bleeding or blood clots.

Fibrinogen in plasma can also be measured by performing the Clauss-test (Undas A, 2017). The terms “Clauss-test” or “CCT”, as used herein, refer to a diagnostic test of a diluted plasma sample, which is submitted to clotting at high concentrations of thrombin.

In a further embodiment, the invention relates to a method wherein the enzyme-based fibrinogen test involves catalytic cleavage by a serine endopeptidase. The term “catalytic cleavage”, as used herein, refers to altering the rate of a chemical reaction of proteolysis by addition of a serine endopeptidase degrading proteins into peptides. A “serine endopeptidase” is an enzyme, wherein serine serves as the nucleophilic amino acid at the active site of the endopeptidase. The “active site” is the region of an enzyme where substrate molecules bind and undergo chemical reaction. The present invention further relates to a method wherein the enzyme-based fibrinogen test involves catalytic cleavage of fibrinogen by snake venom serine endopeptidase, preferably by venombin A. The invention also relates to the method of the invention comprising measuring the proteolytic activity of a serine endopeptidase which is inversely proportional to the fibrinogen level in said sample. The serine endopeptidase acts in a similar manner as thrombin, i.e. by activating fibrinogen and inducing the process of blood clotting. The reaction thus involves conversion of fibrinogen into fibrin. Within the present invention, “inversely proportional” refers to the cause of the decrease of one variable and increase of another variable. Variables refer to proteolytic activity values or fibrinogen level in presence of a serine endopeptidase.

Particularly, the invention is at least in part based on the use of an enzyme-based fibrinogen test that is clot-independent, and where the used enzyme venombin A cleaves fibrinogen by proteolytic activity forming fibrin and causing the release of fibrinopeptide A. In the same reaction, venombin A is also cleaving a synthetic substrate. Thus, the clot-independent fibrinogen test determines the concentration of fibrinogen based on the substrate competition for cleavage by venombin A.

In certain embodiments, the invention relates to the method of the invention, wherein the patient background information comprises or consists of body weight, sex, age and kidney function.

The inventors found that the body weight, sex, age and kidney function are particularly useful to complement the information of the clottability marker and may be used in the classification, prediction, monitoring and/or treatment described herein.

In certain embodiments, the invention relates to the method of the invention, wherein the risk and/or status reference pattern is a machine learning model obtained by training on a dataset of reference subjects and wherein comparing the input data to a risk and/or status reference pattern comprises inputting the input data in the machine learning model.

In certain embodiments, the invention relates to the method of the invention, wherein classifying the risk and/or status of the subject for inflammatory and/or clotting dysregulation event is classifying the risk and/or status of a subject for thrombosis and/or bleeding.

The inventors found that the methods for classification, prediction, monitoring and/or treatment described herein are particularly useful in the context of thrombosis and/or bleeding.

In certain embodiments, the invention relates to a method for prediction of the risk and/or status of a subject for inflammatory and/or clotting dysregulation events, the method comprising the steps of: a) determining a risk and/or status progression indicator by the steps of: i) classifying the risk and/or status of a subject for inflammatory and/or clotting dysregulation events according to the method of the invention during at a first time point; and ii) determining or retrieving a clottability biomarker of a blood sample of the subject at a second timepoint, wherein the clottability biomarker is determined in the blood sample of the subject by at least two different assays; and b) comparing the risk and/or status progression indicator to a prediction reference pattern, wherein the prediction reference pattern is obtained from at least two reference subjects wherein at least one of the reference subjects has previously had an inflammatory and/or clotting dysregulation event and wherein the risk and/or status progression(s) of the reference subject(s) is/are known; c) predicting the risk and/or status of a subject based on the comparison obtained in (b).

The inventors found that the progression of the clottability biomarkers over time, in particular the clottability biomarker comprising or consisting of CCT and TFT, can be used to predict future development of risk and/or status of inflammatory and/or clotting dysregulation events such as thrombosis and/or bleeding by comparison of clottability biomarkers on at least 2, at least 3, at least 4, at least 5, or at least 6 timepoints to reference patterns from population studies.

In certain embodiments, the invention relates to the method of the invention, wherein the prediction reference pattern is a machine learning model obtained by training on a dataset of reference subjects and wherein comparing the risk and/or status progression indicator to a prediction reference pattern comprises inputting the input data in the machine learning model.

In certain embodiments, the invention relates to a method for monitoring treatment response of a subject for inflammatory and/or clotting dysregulation events during treatment, the method comprising the steps of: a) determining a treatment progression indicator by the steps of: i) classifying the risk and/or status of a subject for thrombosis and/or bleeding according to the method of the invention at a first timepoint; ii) determining or retrieving a clottability biomarker of a blood sample of the subject on at least one second timepoint, wherein the clottability biomarker is determined in the blood sample of the subject by at least two different assays; and iii) 1.) an administration timepoint of the anti-inflammatory, anti-platelet, anticoagulation and/or pro-coagulation and/or anticoagulation compound, wherein the administration timepoint is between the first time point and the second timepoint; preferably the administration timepoint and an administered amount of the anti-inflammatory, anti-platelet, anticoagulation and/or pro-coagulation and/or anticoagulation compound; and 2.) a pharmacodynamic response, wherein the pharmacodynamic response is calculated at least based on the clottability biomarker on the first timepoint, the clottability biomarker on the second time point and the administration timepoint, wherein the pharmacodynamic response is calculated at least based on the clottability biomarker on the first timepoint, the clottability biomarker on the second time point, the administration timepoint and the administered amount; b) comparing the treatment progression indicator to a treatment response reference pattern, wherein the prediction reference pattern is obtained from at least two reference subjects wherein at least one of the reference subjects previously underwent treatment with an anti-inflammatory, anti-platelet, anticoagulation and/or pro-coagulation compound and wherein the treatment response(s) of the reference subject(s) is/are known; c) monitoring treatment response of a subject based on the comparison obtained in (b).

Therefore, the methods described herein are useful in monitoring treatment response. This monitoring may be used to amend the treatment regime, the treatment dose and/or the treatment compound. Further the monitoring may be used to determine drug-drug interactions and individual pharmacokinetic and pharmacodynamic.

As such, the method for monitoring described herein may support personalized treatment decisions.

In certain embodiments, the invention relates to the method of the invention, wherein the treatment response reference pattern is a machine learning model obtained by training on a dataset of reference subjects and wherein comparing the treatment progression indicator to a treatment response reference pattern comprises inputting the treatment progression indicator in the machine learning model.

In another embodiment, the present invention relates to a method for providing a personalized anticoagulant treatment regimen of a patient, the method comprising (a) determining clottability of a sample of said patient or providing a known clottability value for said patient; (b) providing a personalized anticoagulant treatment regimen based on the model of clottability obtained by the method of the invention. As used herein, the term “personalized anticoagulant treatment regimen” refers to a plan that specifies the dosage, the schedule, and/or the duration of a clinical intervention applying a set of unit dose(s) that are administered individually to a subject typically separated by periods of time. The term “treatment”, as used herein, refers to a clinical intervention in an attempt to alter the natural course of the individual being treated, and can be performed either for prophylaxis or during the course of clinical pathology. Desirable effects of treatment include, but are not limited to, alleviation of symptoms, diminishing of any direct or indirect pathological consequences occurred by thrombosis or preventing occurrence or recurrence of at least one of the diseases, such as thrombosis, cardiovascular diseases, cancer, stroke or sepsis. The term “patient”, as used herein, refers to a mammalian subject, particularly a human subject afflicted with a disease, preferably thrombosis and/or bleeding, who is likely to benefit from an anticoagulant treatment or an altered anticoagulant treatment. The personalized anticoagulant treatment involves physiological parameters and conditions, pharmacological and clinical treatment information of a patient, and biomarker information, wherein the term biomarker refers to the clottability. Examples of physiological parameters and conditions are age, weight, and sex of a patient.

In a preferred embodiment of the invention, the personalized anticoagulant treatment regimen of a patient is such that if an ongoing anticoagulant treatment of said subject is not sufficient to reduce the attributed risk factor for thrombosis and/or bleeding, the most effective anticoagulant determined in the invention is used for further treatment.

In a preferred embodiment of the invention, the personalized anticoagulant treatment regimen of a patient is such that if an ongoing anticoagulant treatment of said subject is reducing the clottability determined in the invention below a pre-defined clottability interval, the most effective anticoagulant determined in the invention is used for further treatment. Preferably, a “pre-defined clottability interval” refers to a clottability state in which the in vivo thrombin generation due to diseases is reduced to the degree that the risk of thrombosis and/or bleeding is minimal. Based on the modeled clottability using the methods of the invention and thus a most effective anticoagulant treatment, the current treatment may be altered to alleviate such effects (Example 3).

In certain embodiments, the invention relates to the method of the invention, the compound for use of the invention or the method of treatment of the invention, wherein the compound is a compound selected from the group consisting of Vitamin K antagonists, in particular fluindione, warfarin or coumarins, direct thrombin inhibitors, in particular dabigatran, argatroban or hirudin, and direct FXa inhibitors, in particular rivaroxaban, edoxaban, apixaban, heparin, or heparin-like drugs.

In certain embodiments, the invention relates to the method of the invention, the compound for use of the invention or the method of treatment of the invention, wherein the compound is a compound selected from the group consisting of: non-steroidal anti-inflammatory drugs, corticosteroids, rapamycin, high density lipoproteins, HDL-cholesterol elevating compounds, rho-kinase inhibitors, anti-malarial agents, acetaminophen, glucocorticoids, steroids, beta-agonists, anticholinergic agents, xanthine derivatives, sulphasalazine, penicillamine, anti-angiogenic agents, dapsone, psoralens, anti TNF agents, anti-IL-1 agents and statins.

In certain embodiments, the invention relates to the method of the invention, the compound for use of the invention or the method of treatment of the invention, wherein the compound is a compound selected from the group consisting of: irreversible cyclooxygenase inhibitors, adenosine diphosphate (ADP) receptor inhibitors, phosphodiesterase inhibitors, protease-activated receptor-1 (PAR-1) antagonists, glycoprotein IIB/IIIA inhibitors, adenosine reuptake inhibitors, dipyridamole, thromboxane inhibitors and thromboxane receptor antagonists.

In certain embodiments, the invention relates to the method of the invention, the compound for use of the invention or the method of treatment of the invention, wherein the compound is a coagulation factor that promotes clotting and/or reduces bleeding, preferably a compound selected from the group consisting of FVIII concentrate, Alphanate, Humate-P, NovoSeven, Eloctate, Feiba, prothrombin complex, Hemlibra, and tranexamic acid.

In certain embodiments, the invention relates to a storage device comprising computer-readable program instructions to execute the method according to the invention.

The term “storage device”, as used herein, refers to any tangible device that can retain and store instructions for use by an instruction execution device.

In some embodiments, the storage device described herein is at least one selected from the group of electronic storage device, magnetic storage device, optical storage device, electromagnetic storage device, semiconductor storage device, any suitable combination thereof.

A non-exhaustive list of more specific examples of the storage device includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A storage device, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable instructions described herein can be downloaded to respective computing/processing devices from a computer-readable storage medium or to an external computer or external storage device via a network.

Computer-readable program instructions for carrying out operations of the present invention may be assembler instructions, instruction-set-architecture (ISA) instructions, machine instructions, machine-dependent instructions, microcode, firmware instructions, state-setting data, or either source code or object code written in any combination of one or more programming languages, including an object-oriented programming language such as Smalltalk, C++ or the like, and conventional procedural programming languages, such as the “C” programming language or similar programming languages.

The computer-readable instructions may execute entirely on the user's computer, partly on the user's computer, as a stand-alone software package, partly on the user's computer and partly on a remote computer or entirely on the remote computer or server.

In certain embodiments, the invention relates to a server comprising the storage device of the invention, at least one processing device for executing the computer-readable program instructions, and a network connection for receiving the input data.

The term “network connection”, as used herein, refers to a communication channel of a data network. A communication channel can allow at least two computing systems to communicate data to one another. In some embodiments, the data network is selected from the group of the internet, a local area network, a wide area network, and a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer-readable program instructions for storage in a computer-readable storage medium within the respective computing/processing device.

The server may be connected to the device for the acquirement of the vascular image through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable instructions by utilizing state information of the computer-readable program instructions to personalize the electronic circuitry, in order to perform embodiments of the present invention.

In certain embodiments, the invention relates to a system for classification, prediction and/or monitoring a treatment response, the system comprising: a) a measurement setup comprising a container for receiving a blood sample and reagents for determining a clottability biomarker, wherein the clottability biomarker is determined in the blood sample by at least two different assays; b) a processing device for executing the computer-readable program instructions comprising the storage device of the invention and/or a network connection to a server, wherein the server is a server according to the invention; and c) an input and/or retrieval possibility, wherein the input and/or retrieval possibility enables the server and/or the processing device access to the patient background information.

In a further embodiment, the invention relates to a computer system having installed a software able to provide a personalized anticoagulant treatment regimen based on the model of clottability obtained by the method of the invention or parts thereof and a clottability value as input data, preferably wherein the computer system comprises calculating personalized pharmacodynamic values. The “computer system”, as used herein, may take the form of a hardware embodiment, a software embodiment (including firmware, resident software, micro-code, or others) or an embodiment combining software and hardware aspects that may all generally be referred to herein as a circuit, engine, module, or system. Furthermore, aspects of the present disclosure may take the form of a computer program product embodied in one or more computer readable medium(s) having computer readable program code embodied thereon. The program source code may be executed entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer through the internet. A subset of computation or modelling of this invention can also be carried out in the form of internet of things (IoT).

An “installed software” may be executed by the computer system, wherein the software herein may be selected from the group consisting of NONMEM™, Certara Phoenix™ PK/PD software, DoseMe, TDMx, InsightRx, BIOiSIM, Tucuxi, and others. As used herein, the term “input data” refers to the clottability using the method of the invention. As such, the term “input data” refers to the clottability information obtained from the diagnostics of the biomarker alone or in combination with further data such as patient background data. Example 3 describes three modules for the design of a personalized anticoagulant treatment system, wherein module 1 is the information system for patients which requires manually or automatically obtained input. Module 2 determines fibrinogen levels using the CCT and TFT test, in order to generate statistical estimations about fibrinogen level, clottability state, thrombin and/or fibrinolytic activity. Module 3 may issue warning regarding drug-drug interaction and provide a personalized anticoagulant treatment regimen. The computer system or software may execute all or part of the methods of the invention. Generally, the modules described herein refer to logical modules that may be combined with other modules or divided into sub-modules despite their physical organization or storage.

Accordingly, the invention is at least in part based on the surprising finding that a computer system may be able to provide a personalized anticoagulant treatment regimen based on the model of clottability and a clottability value as input data. The determined personalized anticoagulant treatment regimen may be the output data providing management guidance on precise and personalized anticoagulant dosage for reducing the risk and occurrence of thrombosis and/or bleeding.

The general methods and techniques described herein may be performed according to conventional methods well known in the art and as described in various general and more specific references that are cited and discussed throughout the present specification unless otherwise indicated. See, e.g., WO2019/068940, Mackie et al. 2002, Mackie et al. 2003, Undas A. 2017, Zirlik and Bonde 2016, Kajy and Ramappa 2009, Adeboyeje et al. 2017, Ordi-Ros et al. 2019, Harter et al. 2015, Hempenius et al. 2021, Brown et al. 2020, Hori et al. 2013, Koverech et al. 2018.

While aspects of the invention are illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below. The invention also covers all further features shown in the figures individually, although they may not have been described in the previous or following description. Also, single alternatives of the embodiments described in the figures and the description and single alternatives of features thereof can be disclaimed from the subject matter of the other aspect of the invention.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. “preferably” means one option out of a series of options not excluding other options. “e.g.” means one example without restriction to the mentioned example. By “consisting of” is meant including, and limited to, whatever follows the phrase “consisting of.”

### BRIEF DESCRIPTION OF DRAWINGS

FIG. 1: Comparison between determined plasma fibrinogen concentrations at different time points obtained from CCT (squares) and TFT (triangles) tests, and the derived clottability (sphere and solid line) in a healthy subject (A) and a patient suffering from chronic hyperclottability (B). The healthy subject (A) has a normal-clottability that fluctuates between 0 and 0.3, whereas the patient (B) displays high clottability (hyperclottability) that fluctuates between 1.7 and 2.5 within the time period.

FIG. 2: Example of a typical PK (pharmacokinetic) model of a novel oral anticoagulant (e.g. rivaroxaban). The Y-axis represents the drug concentration, while X-axis indicates the progression of time. The peak plasma concentration of the anticoagulant reaches peak level shortly after intake of the oral anticoagulant. Each “peak” to “trough” phase is equally divided into three phases (top, middle and bottom), demarked by three brackets.

FIG. 3: Comparison between plasma fibrinogen concentrations obtained from CCT (squares) and TFT (triangles) tests at different time points and the derived clottability (sphere and solid line) in a healthy subject which had developed an acute inflammation before day 0. The clottability within four days ranges between 1.2 and −0.6. The clottability is being reduced within two days signifying the inactivity of thrombin and subsequent increased activation of fibrinolysis that generates high concentration of anti-coagulable factors such as FDPs, which interferes with the CCT test.

FIG. 4: Example of a pharmacokinetic model, which illustrates the PK profile of an anticoagulant before dosage adjustment (labeled by the horizontal arrow bar below the X-axis). Y-axis represents the drug concentration and X-axis represents the progression of time. Based on the clottability test, the coagulation pathway is still more active than it should be and exhibits hyperclottability. To reduce the hyperclottability, module 2 calculates the needed reduction and the new therapeutic range, based on a pharmacodynamic (PD) model. The PD model proposes adjustment of the therapeutic range (demarcated by the two dotted “max” and “min” lines) to achieve ideal clottability. The new therapeutic range between “max” and “min” is overlaid to the simulated PK profile, wherein the newly adjusted therapeutic regimen is adopted at the indicated time (vertical solid arrow indicates the start of the new regimen).

FIG. 5: Fibrin(ogen) determined via CCT and TFT within 2 different populations: control healthy vs liver disease patient.

The following are examples of methods and compositions of the invention. It is understood that various other embodiments may be practiced, given the general description provided above.

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those skilled in the art that the techniques disclosed in the examples which follow represent techniques used in the present invention to function well in the practice of the invention, and thus can be considered to constitute preferred modes for its practice. However, those skilled in the art should appreciate, in light of the present disclosure that many changes can be made in the specific embodiments which are disclosed and still obtain a like or similar result without departing from the spirit and scope of the invention. Several documents including patent applications, manufacturer's manuals and scientific publications are cited herein. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### EXAMPLES

### Example 1

For determination of the clottability of blood, combined clottability assays were performed using a (a) clot-based and (b) enzyme-based fibrinogen test. The information provided by the assay is whether the blood is normal-clottable, hyperclottable, or hypoclottable by measuring fibrinogen concentration in blood plasma.

To estimate the clotting dynamics, the fibrinogen concentration obtained by the clot-based assay was subtracted from the concentration obtained by the enzyme-based fibrinogen assay. Thus, clottability is determined by deduction of the CCT (clot-based Clauss fibrinogen test) value from the TFT (true fibrinogen test) value (clottability=CCT−TFT). Exemplary, high values after subtraction of the obtained concentrations of the tests indicate high amounts of pro-coagulable factors and thus increased thrombin activity. Therefore, the greater the in vivo thrombin activation, the higher the obtained values from the clottability test. This leads to the finding that high values relate to an increased clottability.

### a) Clot-Based Fibrinogen Test

Clot-based fibrinogen tests have been described in by Mackie et al. (2002). According to the invention any of the following tests from a selection of tests i.e. CCT (clot-based Clauss fibrinogen test), PT (prothrombin-time-derived fibrinogen test) or PTT (determination of partial thromboplastin time), or any other clot-based test can be performed to obtain the fibrinogen level.

The skilled person is aware of how to perform the CCT test. Thus, as an example, Multifibren-U from Siemens and/or HemoslL Fibrinogen-C from IL/Werfen can be used. Moreover, it is known that the CCT test can show a higher fibrinogen concentration than the actual concentration due to enhanced clotting efficiency by the pro-coagulable factors due to the thrombin activity.

Besides the use of blood plasma of individuals, essential reagents for the CCT test may be:
serine protease (e.g. thrombin),
an agent that prolongs polymerization time (e.g. Gly-Pro-Arg-Pro or Gly-Pro-Arg-Pro-Ala or similar),
heparin neutralizing agent (e.g. polybrene).

As an example, another clot-based fibrinogen test, which can be used within the scope of the invention is the PT test (e.g. PT-fibrinogen test from IL/Werfen) or PTT test. The PT test is based on the change of scattered light or optical density, wherein the commercial availability and composition of selected reagents as well as the protocols to perform the assays can be variable and adjustable.

Besides the use of blood plasma of individuals, essential reagents for the PT or PTT test may be:
thromboplastin reagent (e.g. rabbit brain thromboplastin),
heparin neutralizing agent (e.g. polybrene).

### b) Clot-Independent Enzyme-Based Fibrinogen Test

Clot-independent enzyme-based fibrinogen tests have been described in WO2019/068940. The fibrinogen test specifically determines the fibrinogen concentration even in presence of natural and physiological interfering factors, wherein factors can be pro- and/or anti-coagulable. The assay was performed using the TFT test, e.g. the Pefakit fibrinogen test from Pentapharm.

Besides the use of blood plasma of individuals, essential reagents may be:
serine endopeptidase (Venombin A, e.g. Batroxobin),
peptide substrate (e.g. Pefachrom TH Tos-Gly-Pro-Arg-pNA),
polymerization inhibitor (e.g. Pefabloc FG Gly-Pro-Arg-Pro),
inhibitors for unspecific proteinase activity (e.g. Pefabloc SC AEBSF, Aprotinin),
chelating agent (e.g. EDTA).

Fibrinogen can be activated through its catalytic cleavage by a thrombin-like enzyme serine endopeptidase from snake venoms. This assay determines the fibrinogen concentration in plasma samples by competitive enzyme kinetics.

### Example 2

The clottability test can provide an indication of thrombin production. Adopting the method which incorporates data of patients and a novel biomarker can help manage the anticoagulant treatment. Three clinical studies were performed on healthy individuals and on patients receiving anticoagulant treatment.

a) Clinical Study A: Calculating Clottability Value of a Healthy Population with Reduced Levels of Inflammation

Clinical study A was carried out in healthy individuals (n=9). Plasma samples were collected for measuring clottability and determining the CRP (C-reactive protein) level. The health status of these individuals was confirmed obtaining low levels of CRP, indicative for reduced levels of inflammation which may indirectly correlate with the activity of the coagulation pathway. However, high levels of CRP indicate acute inflammation.

### CRP (C-Reactive Protein) Assay

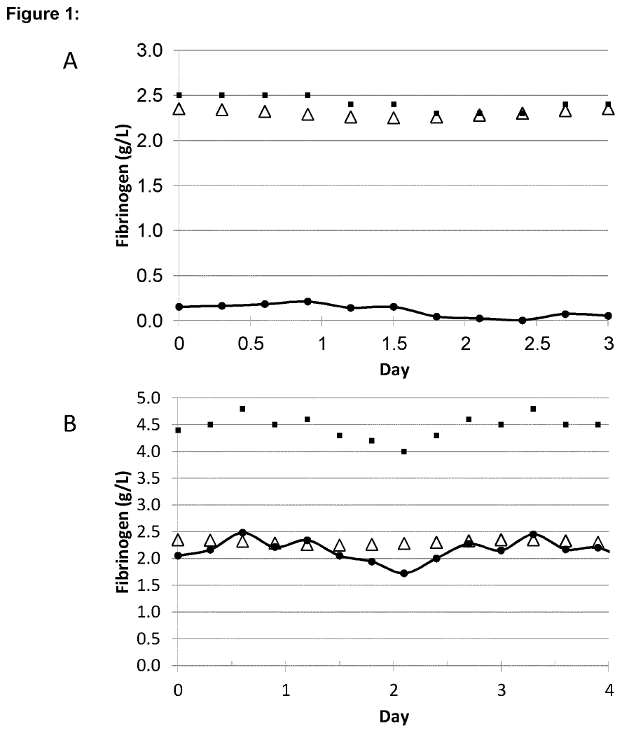

The CRP assay is making use of antibody-antigen binding, wherein CRP is a common inflammatory biomarker and can be measured by means of ELISA. Upon immune response the biomarker can induce the expression of a tissue factor (TF), which can further activate the coagulation cascade. The assay can also examine the health status of an individual, as used thereof in clinical studies. Healthy individuals have a normal clottability ranging between −0.5 and 0.5. These low values are indicative for low coagulation pathway activity correlating with reduced inflammation (FIG. 1A).

### Results

Exemplary, in the clinical study A, measured CRP levels fluctuated between 21 and 300 ng/mL. These values were lower than the reference level of 5,000 ng/mL. The determined clottability values (n=9) were ranging between −0.3 and 0.3 indicating a reduced inflammation. Whereas plasma with a CRP value of >7,000 ng/mL showed clottability of 1.7 and was used as a control indicating increased inflammation.

This study indicates that the clottability range for the healthy population mostly lies between −0.5 and 0.5 (the widening of 0.2 is the inclusion of possible errors). This range of clottability represents normal clottability, i.e. values ≥−0.5 and ≤0.5, wherein low fluctuation of approximately “0” (zero) signifies low coagulation pathway activity and correlates to reduced inflammation (FIG. 1A).

### b) Clinical Study B: Calculating Clottability Value of a Healthy Population

To further confirm the clottability values of a healthy population, clinical study B was performed. Therefore, for the clinical study, a random population of self-proclaimed healthy individuals (n=36) and their plasma samples were included in order to measure the clottability value and determine the clotting state (hyperclottable, hypoclottable, or normal-clottable).

### Results

The determined clottability of the tested individuals of the clinical study fluctuated between −1.1 and 1.5. Half (50%) of the investigated population had normal clottability values ranging between ≥−0.5 and 50.5 and the other half encompassed values outside the range of −0.5 and 0.5. Whereas, 28% of the population had clottability values ranging between −1.5 and −0.5, and 22% of the population had values between 0.5 and 1.5.

The negative clottability (values >−1.5 and <−0.5), or hypoclottability (represented by 28% of the population), reveals the production of fibrinolysis-induced fibrin degradation products (FDPs) or fibrin-derived anti-coagulable factors, which are inhibiting the clot formation. This indicates also activation of the coagulation pathway at an earlier time point. Once these anti-coagulable factors are further processed to later stages, they can lose their negative effect in the clotting process.

On the other hand, the positive clottability (values >0.5 and <1.5), or hyperclottability (represented by 22% of the population), indicates notably more pro-coagulable factors due to the activation of the coagulation pathway. This study provides an initial reference range for a healthy population. Since clottability within an individual is changing within hours. Therefore, to be able to create a more accurate computational model of an individual and compare this model to populational models in the database, the clottability test can be performed multiple times at different time points.

### c) Clinical Study C:

### Calculating Clottability Value of Patients Receiving Anticoagulant Treatment

Clinical study C was initiated to survey the modern anticoagulant treatment against thrombosis and thus to evaluate the efficiency of the treatment by measuring the activity of the coagulation pathway. Plasma samples from 19 patients were tested to investigate the coagulation pathway activity using the clottability test (CCT and TFT) and D-dimer test. These patients were receiving various anticoagulant drugs, such as rivaroxaban, apixaban, edoxaban, dabigatran, or argatroban, and the drug concentrations of each individual were determined. The aim of the study was to examine the activity of the coagulation pathway by means of both, the clottability test and D-dimer test in relation to the determined drug concentration in the plasma.

### Coagulation Factor Assay: D-Dimer (DD) Assay

The D-dimer assay is an immunoassay based on an antibody-antigen binding technique. D-dimer is an antigen derived from the proteolytic degradation of fibrin. The assay is an in vitro diagnostic test indirectly indicating thrombin or coagulation pathway activation. During fibrinolysis, blood clots are being proteolytically degraded leading to the formation of fibrin-derived intermediates, such as fibrin degradation products (FDPs) and D-dimers (DDs). The DD concentrations could indirectly indicate in vivo thrombin activity, which could occur hours to days earlier. By means of the D-dimer assay, various plasma components like human anti-mouse antibody and FDPs interfere. Thrombosis or high in vivo thrombin activity lead to high DD values. Thus, the DD assay is mostly used to verify if a patient is experiencing thrombosis.

### Results

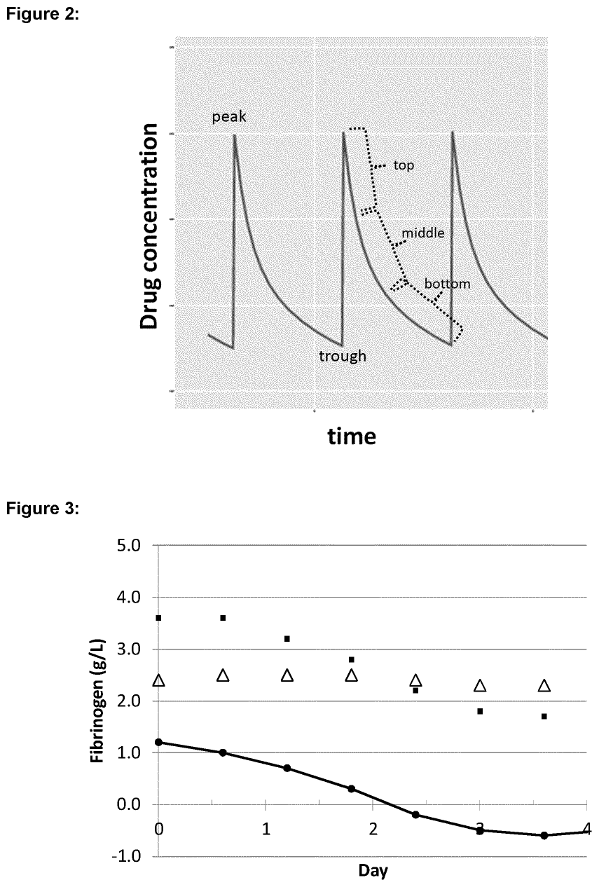

The pharmacokinetics (PK) of the anticoagulant in plasma normally exhibits a very sharp rise of drug concentration reaching the peak level, hence peak concentration within a short period of time after anticoagulant drug intake. The drug concentration in plasma slowly diminishes from the peak (top) concentration reaching the trough (bottom) concentration, very close to the lowest detectable concentration (FIG. 2). The pharmacokinetic (PK) profile correlates to the drug concentrations, which are classified into three levels (top, middle, bottom), by equally subdividing the peak to trough concentration into three parts (FIG. 2, Table 1).

The DD test can show negative and positive values, wherein values below 500 are classified as negative and values greater than 500 are classified as positive. Values ranging between 501-1,500 are denoted as “+” and indicate weak positivity. Whereas a medium positivity (denoted as “++”) has values between 1,501-2,500 and a strong positivity (denoted as “+++”) has values greater than 2,501 (Table 1).

Further, there is a correlation between the results of the clottability test and DD test, even though they are detecting different analytes. About 60% of the patients showed positive DD values when treated with anticoagulants, indicating activation of the coagulation pathway. Additionally, the negative values of the DD test result did not indicate inactivity of the coagulation pathway, because if the blood sampling time was not at the DD peak phase or not at the right time, the measurement would result in negative findings. In fact, patients with ID NOs: 11, 12, 13, 16, 18 and 19 showed coagulation pathway activation based on the clottability test, while the values of the DD test were negative. It is suggested that this happens, because the D-dimer forms at a later time point after the activation of the coagulation pathway. Similar to DD, the clottability biomarker is dynamic within individuals over different time periods according to the health status and anticoagulant treatment. In contrast to DD, clottability can be measured in an earlier point of time giving faster results. Clinical study C was performed to evaluate the clottability test in direct comparison with the DD test when patients were treated with anticoagulants at one single time point. The study confirmed that optimization of anticoagulant dosage is needed individually to reduce the risk of ineffective treatment. Thus, the clottability test can be used to enable personalized monitoring and treatment adjustment, and hence effective treatment.

TABLE 1
Results of clinical study C (participation of 19 individuals
(ID)) comparing the PK profile, the measured clottability and
the D-dimer values after administration of an anticoagulant.

| ID | Anticoagulant | PK profile | Clottability test [g/L] | DD test |
|---|---|---|---|---|
| 1 | apixaban | middle | 0.8 | + |
| 2 | apixaban | middle | 0.8 | ++ |
| 3 | apixaban | bottom | 1.8 | ++ |
| 4 | apixaban | bottom | 2.7 | +++ |
| 5 | argatroban | bottom | 2.7 | + |
| 6 | argatroban | bottom | 2.1 | + |
| 7 | argatroban | bottom | 2.8 | ++ |
| 8 | argatroban | bottom | 3.8 | ++ |
| 9 | argatroban | bottom | 1.8 | ++ |
| 10 | dabigatran | bottom | −0.3 | ++ |
| 11 | dabigatran | middle | 1.3 | − |
| 12 | dabigatran | bottom | 1.2 | − |
| 13 | edoxaban | middle | −0.6 | − |
| 14 | edoxaban | top | 1.7 | ++ |
| 15 | rivaroxaban | top | 1.7 | + |
| 16 | rivaroxaban | bottom | 1.7 | − |
| 17 | rivaroxaban | top | −0.5 | − |
| 18 | rivaroxaban | middle | 0.6 | − |
| 19 | rivaroxaban | bottom | 2 | − |

Based on Table 1, about 60% of all tested samples exhibited clottability >1.5, and about 40% of these samples had clottability within the range of the healthy population of the clinical study B. The higher the PK profile (top) and thus the drug concentration, the greater the inhibition of the coagulation pathway. A PK profile at the bottom means that the drug concentration in the plasma is nearly at the lowest point and such a condition is less capable of inhibiting the activation of the coagulation pathway. High clottability values signify high activation of the coagulation pathway. Exemplary, plasma samples of patients with ID NOs: 3 and 4 receiving apixaban had a PK profile at the bottom phase and the clottability values were greater than 1.5, hence the plasma was hyperclottable in comparison to the plasma of the healthy population. This means that for these two patients the coagulation pathway was activated. The high DD levels of these patients confirmed the coagulation pathway activation and the followed fibrinolysis. This shows, the importance of a personalized drug treatment to adjust the dosage, since, as shown in the example, specifically for these two patients the coagulation pathway activation needs to be reduced.

In another example, a patient group received the anticoagulant rivaroxaban. Blood samples of patients with ID NOs: 15, 16 and 19 showed hyperclottability (clottability >1.5) at PK profiles of top and bottom phases, which was indicative of high coagulation pathway activity. The plasma samples of patients with ID NOs: 17 and 18 was normal-clottable (sec clinical study A), indicating low level of in vivo coagulation pathway activity at the time of blood sampling and anticoagulant treatment regimen. Consistent observations were seen in patients treated with edoxaban, dabigatran and argatroban.

### Example 3

### The Concept of the CPT System

The CPT (clottability-based personalized treatment) system is a novel clottability biomarker guided anticoagulant management system. Examples of such a CPT system are:
1. a web-based application, that accepts information (including clottability data) which manually enter into the system and produce a list of calculated results.
2. an application installed to a computing device of any operating system such as IoT, etc, which accepts information (including clottability data), wherein the information may enter manually into the system and produce a list of calculated results.
3. a system coupled with a device capable of measuring the clottability and other biomarkers using blood samples such as IoT, etc. The data produced by the device is automatically transferred into the build-in computing systems or transmitted through wired or wire-less connections to a computer system. A list of results is produced to guide physicians and clinicians by providing personalized and optimized anticoagulant treatment regimen.
4. computer systems having partially or completely such functions as described in 3., which functions can be transmitted into a USB stick.

In order to perform health and risk assessments, simulations and calculations using the pharmacodynamic (PD) and pharmacokinetic (PK) models are required. Such examples of parameters and information are:
1. biomarker information obtained from the clottability test and information of each test, CCT and TFT, separately,
2. physiological parameters of patients, such as age, sex, physiological conditions (e.g. blood pressure, kidney function), or illness,
3. pharmacological and clinical treatment information regarding anticoagulant, anti-platelet, anti-cancer and anti-inflammatory drugs.

The CPT computer system may consist of three build-in modules. Additionally, examples of compartments to extent such a CPT system may be a remote device (e.g. IoT device) for patients, which allows parameters for e.g. blood pressure data transmission or transfer of other data of important parameters. This system may inform practitioners about any unusual health event of a patient. Such remote system may also possess build-in compartments of performing the clottability test, providing a test analysis of health status and risks, PD/PK model or anticoagulant treatment regimen. The results than may be transmitted to a server.

Further, the CPT system-build-in-computational models may be created by machine learning approaches or traditional approaches, such as Bayesian-based systems and/or any statistical PK modelling program, well-known by skilled person.

### Module 1

Module 1 is described as the information system for patients, which requires manually or automatically obtained input. Thus, this module is designed to be the entry point of information by automatically getting the generated patient information or recorded information by other computer systems, e.g. LOINC© Mapping systems. Module 1 is also designed to serve as an information storage system of patients, which information as well as calculations and models obtained from module 2 and 3 are stored within this module 1. Thus, the modules are cooperating and exchanging information between modules and electronic medical record systems. Module 1 is also designed to be accessed or to interact with existing electronic health/medical record systems, so that patient derived information can be securely exchanged. Another key function of module 1 is its management system of patient database, such as scheduling appointments for visits, consultation, or blood sampling etc. This can remind patient about drug intake and appointments for doctor visits.

### Module 2

Module 2 acquires information from module 1, produces modelled clottability data by means of computational analysis and a personalized therapeutic range by means of computational models. This module may be simulating clottability models based on individual clottability information over a time period. In order to produce mathematical models for health and risk assessments, the individual clottability data is rated and modeled statistically according to the profiles of the population. Individual basal value, intra- and inter-individual variations of the pharmacodynamic effect can be determined based on a combination (e.g. subtraction or division) of the clottability read outs of at least two different clottability assays (sec Example 9).

The clottability conditions indicate any activity regarding the coagulation pathway and immune response, and can thus produce a health status and risk assessment toward thrombosis and bleeding. The health status and risk assessment can be calculated statistically based on populational studies. Acquired data from patients, which can give information about the health condition, may be for example:
fluctuating thrombin and fibrinolytic activity,
clottability state acquired from clottability test.

Computational analysis in risk assessment studies about thrombosis and/or bleeding, and statistical analysis may be performed within module 2. The acquired data from module 1, may be analyzed in module 2 in order to investigate the health condition of an individual by using for example information such as:
thrombin and fibrinolytic activity,
clottability state acquired from the clottability test,
population studies,
determined fibrinogen levels obtained from the CCT and TFT test,
PK and PD profile simulation and alignment during anticoagulant drug (alone or together with other drugs) intake,
   calculated plasma drug concentration.

For example, if a patient is being prescribed with only one anticoagulant drug, module 2 is able to calculate whether this anticoagulant with a prescribed concentration and regimen improved the state of clottability. If the patient is being prescribed with multiple medications at a time such as anti-platelet, anti-inflammatory and/or anticoagulant drugs, module 2 may be able to recommend stepwise reduction of hyperclottability and anticoagulant drug concentration adjustment for new drug prescription. Within this module a multiple medication scheme may be generated to ensure effectiveness of an on-going treatment. Then, additional clottability data will be needed to calculate the following anticoagulant dosage level. Another example is, if a patient is suffering under a coagulation factor deficiency (acquired or hereditary) during therapy with an anticoagulant, the system may warn the user and recommend reducing the therapeutic range or use a stepwise approach depending on the case.

### Results

FIG. 3 demonstrates a healthy individual developing an acute inflammation and elevated thrombin activity before day 0, which diminishes before day 2. Whereas, fibrinolytic activity, mainly mediated by plasmin activity, is increasing before day 2. Thus, predominant presence of anti-coagulable factors such as FDPs affects the clotting process of the CCT test. Hence, one feature of module 2 is to generate models of in vivo fibrinolytic activity based on the clottability data.

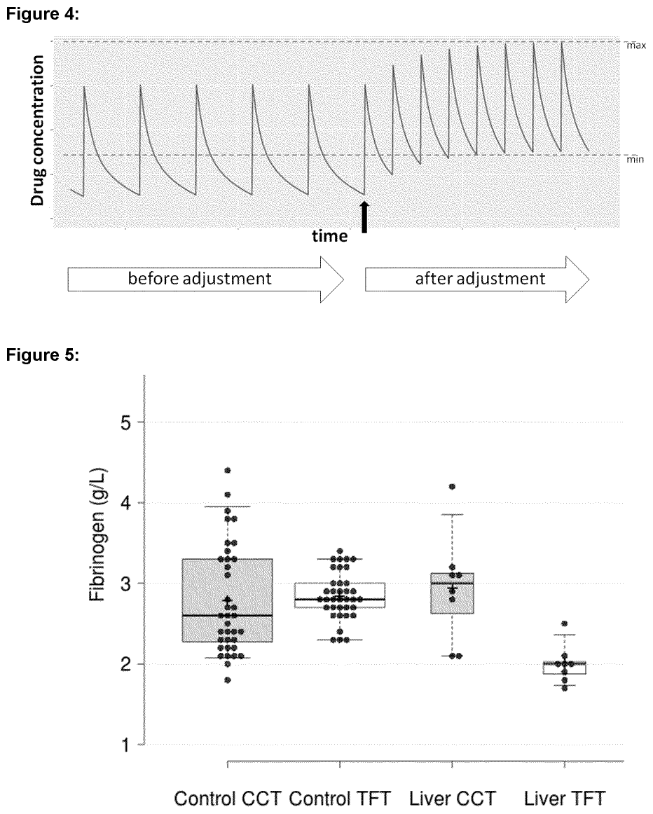

Module 2 calculates the plasma concentration of an anticoagulant or a series of anticoagulants, predicted to achieve the adjustment of clottability to an acceptable level. This concentration range, which contains the minimal and the maximal drug concentrations, or other PK parameters appropriate to describe the relationship between individual drug exposure and clottability adjustment, is calculated based on mathematical and/or statistical models generated from clinical data of the patients treated with an anticoagulant. Within this concentration range, the patient will have safe and highly effective treatment. This method is applicable to patients already receiving anticoagulant treatment. According to the clinical studies, patients with ID NOs: 15, 16 and 19 taking rivaroxaban are showing hyperclottability when the PK is at the top and bottom levels, respectively. However, clottability data of patients with ID NOs: 17 and 18 are showing effective treatment and normal clottability values after intake of the same dosage of rivaroxaban. As illustrated in the example of FIG. 4, the rivaroxaban treatment regimen and dosage could be adjusted individually for patients with ID NOs: 15, 16 and 19. After accomplishment of treatment adjustments of these patients (ID NOs: 15, 16 and 19), clottability should be improved as exemplified by patients with ID NOs: 17 and 18. The same principle can be applied to patients prescribed with apixaban (Table 1, ID NOs: 1 to 4) or any other anticoagulant.

The safety threshold of each anticoagulant is contained in the CPT systems. Module 2 acquires the patient's details stored in module 1 and analyzes the interactions with other prescribed drugs, e.g. anti-platelet on the risks of adverse events and recommends a stepwise approach to anticoagulant regimen adjustment. For the sake of illustrating one of the safety features, a patient who needs only anticoagulant treatment, is recommended to receive the drug treatment concentration approaching the safety threshold of this anticoagulant. The system is able to warn the user and recommend a lower and safer dosage in combination with other medications such as regulators of inflammation or anti-inflammation. Therefore, CPT systems enable multiple medications to achieve safer and effective treatment.

### Module 3

The PK modelling is a key feature of module 3, which integrates the results of computational and statistical analysis performed by module 2 and the parameters relevant to personalize PK simulation models by customizing the best anticoagulant treatment regimen for an individual, and providing simulated treatment regimen. There is already a wide range of existing software for modelling PK and personalized dosage and treatment regimen in order to provide data about diseases and medications. However, the current CPT systems are able to translate the target treatment effect of an anticoagulant to its concentration in PK and recommend a personalized drug dosage and regimen according to parameters contained in module 1. Another feature of module 3 is the ability to analyze the gap between personalized and standard one-size-fit-all posology. It can also compute the risks due to the gaps allowing the user to understanding and managing the possible risks. Further, module 3 can issue warning regarding drug-drug interactions and produce revised simulation of the personalized PK when the patient is receiving for example P-glycoprotein inhibitor or inducer. CPT systems are able to simulate the PK with consideration of possible genetic variations in metabolic enzymes that influence PK of individual anticoagulants.

### Results

Exemplary, the PK model may be influenced by drug-drug interactions. The drug absorption of orally administrated anticoagulants is affected by P-glycoprotein inducers (e.g. Rifampicin) and inhibitors (e.g. Verapamil), and may influence the drug PK profile. In case of co-administering Rifampicin and anticoagulant drug dabigatran, the maximum plasma concentration of dabigatran may decrease by approximately 67%, which may significantly impact the anticoagulant treatment efficacy. Another example of drug-drug interactions is the co-administration of cytochrome P450 inhibitors and oral anticoagulants. Since the PK model is affected by drug-drug interactions and many other parameters, module 3 takes all these into consideration and recommends drug dosage and treatment regimen, warnings and risks of drug-drug interactions, and possibility of a step-wise approach to reduce clottability to the target interval, to achieve reducing the risks of thrombosis and/or bleeding.

### Example 4

Liver disease is an interesting study model for coagulation system, due to the fact that liver produces most coagulation factors, including both pro- and anti-coagulation factors and other proteins involving fibrinolysis. As liver disease progresses, the coagulation system is going through significant changes due to the re-balancing of these factors and hence the typical coagulation lab parameters are incapable of providing insights into the coagulation system. These current lab parameters are not able to predict thrombosis and/or bleeding risk of such patients. Hence, there is a need for new diagnostics for such prediction. This example demonstrates the use of clottability parameters in assessing the risks of thrombosis and bleeding.

This novel biomarker clottability is composed of several key components, CCT, TFT and CCT:TFT. The diagnostic method TFT is important as it gives the measurement of true fibrinogen level in the blood sample. It is so specific that it does not measure any molecule very similar to fibrinogen, such as the fresh fibrin derivatives generated by the removal of fibrinopeptide Aa from fibrinogen through the action of thrombin, the so-called in vivo thrombin generation.

On the other hand, CCT gives a mixture of indications regarding the patient at the time of blood sampling. These indications include the amounts of fibrinogen and fibrin-derivatives. These fibrin-derivatives are present in the blood due to the trauma- or disease-induced in vivo thrombin generation. These derivatives exist in various dynamic manners as different species are generated, evolved, transformed, degraded at different time since the initial action of thrombin on fibrinogen and subsequent modification and breakdown through fibrinolytic system. These derivatives exert different effects on the clotting reaction that CCT is dependent on, either enhancing clotting (e.g. soluble fibrin complex), inhibiting clotting (e.g. some FDP s) or neutral effect (e.g. DDs). Therefore, CCT is a method that gives measurement of fibrinogen and fibrin-derivatives. CCT is able to be responsive to the fast in vivo thrombin generation, which DDs is not capable of. Additionally, the fibrin-derivatives detectable by CCT are of very appropriate half-lives, in comparison to inappropriately long half-life of DDs.

The total effect of these derivatives is easily measured by determining the relationship between CCT and TFT. For simplicity reason, this patent mainly exemplifies the relationship with simple arithmetic such as CCT subtracted by TFT. When the CCT has a value greater than TFT (e.g. FIG. 1B), this signifies that the total effect exerted by these derivatives are enhancing clotting reaction and the predominant derivatives are for example soluble fibrin complexes. Therefore, the most important information regarding in vivo thrombin generation is embedded in CCT. The value of CCT thus signifies the health condition.

TABLE 2
Clinical samples of chronic liver disease patients were measured
for several parameters, such as factor V level (% of normal
level) and other fibrin(ogen) and derivative such as DD.

| | |
|---|---|
| Liver function | |
| (based on | Fibrin(ogen) |

| ID | FV %) | CCT | TFT | DD |
|---|---|---|---|---|
| 1 | 50 | 3.1 | 2 | neg |
| 2 | 50 | 3.1 | 2 | pos |
| 4 | 56 | 2.9 | 2.5 | pos |
| 5 | 46 | 2.1 | 2 | pos |
| 6 | 45 | 4.2 | 1.7 | pos |
| 7 | 30 | 2.1 | 2.1 | pos |
| 8 | 66 | 2.8 | 1.8 | pos |
| 9 | 48 | 3.2 | 1.9 | pos |

The chronic liver disease plasma samples were collected and analyzed for factor V and DD. The samples were also measured with CCT and TFT. The factor V, a pro-coagulation factor, was determined to estimate the liver functional level. These patients displayed abnormal standard coagulation lab parameters, e.g. prolonged PT and aPTT, albeit normal fibrin(ogen) level, determined via standard CCT. The fact that these patients have about on average 50% of factor V compare to normal, the patients were at advanced stage of liver disease. These patients have on average TFT of about 2, which is statistically and significantly lower than the mean of 2.8 (FIG. 5). With such disease condition, the risks of thrombosis and bleeding are not to be put into the same category as patients having no liver problem, and no reduced levels of both pro- and anti-coagulation factors. The thrombotic risk of such liver patients is higher. For example, a liver disease patient has CCT of 4 and TFT of 2, hence the clottability (or CCT:TFT) of +2. This patient will have a greater thrombotic risk than a patient without liver disease, who has CCT of 5, TFT of 3 and CCT:TFT of +2. Therefore, TFT is able to give strong indication of how the pro- and anti-coagulation factors re-balance themselves beside true fibrinogen level, as exemplified by liver disease here. Algorithms which able to predict the risk are created either through machine learning of many clinical cases or other mathematical means.

The TFT fibrinogen levels of healthy population distribute between 2.3 and 3.4, with average at 2.7 (Control group in FIG. 5). Only in severe liver disease population can the TFT fibrinogen goes below the normal level (Liver group in FIG. 5). Therefore, unlike CCT which reflects the disease situation and the pathological in vivo thrombin generation, TFT value of any individual, either healthy or sick, will distribute within these 2 populations represented in FIG. 5. This further confirmed with a group of patients treated with anticoagulants (Table 1), whose TFT values lay between 1.9 and 3.2. Since TFT is lacking the function of showing fast dynamic in vivo thrombin generation for individual, the further examples illustrate the drug treatment effects on hyperclottability reversal or reduction via changes only in CCT values, for clarity and simplicity reason. Another important point, the individual (ID 6) with less than 50% liver function can never produce fibrinogen above normal level e.g. greater than 4 g/L (Table 2). Hence, CCT is reflecting more than fibrinogen level, it is an indicator of fibrin-derivatives produced by in vivo thrombin generation. The use of biomarker clottability, particularly the CCT component, is most of all important to reveal the underlying disease conditions and the effects of pharmacological interventions. The component CCT is the only biomarker component that reveals the pathological in vivo thrombin generation, which is the root cause of hyper-clottability or hypercoagulability in many diseases, since CCT is able to detect the generation of fibrin-derivatives. Therefore, when a clinical study designed to measure the general trend of CCT after pharmacological interventions known to interfere in the coagulation system, this study is able to demonstrate if CCT, the dynamic component of clottability biomarker, can be generalized in its utility as a biomarker to monitor disease and drug treatment progressions.

### Example 5

In order to study the effect of anticoagulants on CCT, clinical studies were performed and clinical data were collected and analyzed to illustrate the clinical utility of CCT in indicating the dampening of coagulation system via pharmacological interventions. Several studies, conducted in accordance to the medical research ethical standards, were performed to conclude such clinical utility of CCT in modulating hypercoagulability or hyperclottability caused by disease.

Firstly, anticoagulant studies were performed on healthy population to show that healthy individuals have little fibrin-derivatives or absence of hyperclottability and normal CCT values, and the fibrin-derivative-reducing anticoagulant-treatment in healthy individuals does not significantly change the CCT values. In such study, rivaroxaban study was performed on 6 healthy volunteers, with ages between 18-50 years. They fulfilled the criteria of normal vital signs and lab screening tests for infection, hematology and coagulation, and had no abnormalities at physical examination. They were given 15 mg rivaroxaban twice daily for 2.5 days. The dosage of twice-daily 15 mg was chosen as it is the highest approved dosage for the initial treatment of acute venous thromboembolism (VTE). The apixaban study was performed on another 6 healthy volunteers, with the same health profile requirements as rivaroxaban study. These 6 volunteers were given 10 mg apixaban twice daily for 3.5 days. Blood samples were drawn just before anticoagulants intake and 3 hrs after the last intake. Measurement of fibrinogen was in duplicate.

In rivaroxaban study, CCT changed from average of 2.75 (with standard deviation 0.92) before rivaroxaban treatment to average of 2.64 (with standard deviation 0.93) after treatment. The difference of −0.11 is not statistical significant. The changes in CCT before and after apixaban treatment were from average of 2.56 (with standard deviation 0.36) to average of 2.38 (with standard deviation 0.25). The difference of −0.18 is not statistical significant either. These individuals generally have their CCT values around the healthy value range, indicating they are generally absent of abnormal amount of fibrin-derivatives, which are generated by pathological in vivo thrombin generation, which gives rise to hyperclottability such as e.g. severe COVID-19 patients can have CCT values greater than 6.0 and thrombosis. In another word, the volunteers were healthy and had no hyperclottability due to their normal CCT values. Additionally, even when their coagulation system was highly inhibited, their CCT values did not significantly change. These studies indicate conclusively that pathological in vivo thrombin generation is generally absent in healthy individuals and high dose of pharmacological inhibition of coagulation system in such healthy individuals produces insignificant clottability change as revealed by CCT.

Secondly, anticoagulant studies were performed on patients suffered from thrombosis to show that patients with high fibrin-derivatives or hyperclottability were helped by the inhibition of coagulation system, with the concomitant reduction of the fibrin-derivatives revealed by CCT. The study revealed the treatment efficacy through correlation with the reduction of CCT values and the improvement of physiological function.

Seventy patients who had complete clinical record and were diagnosed with acute cerebral infarction (ACI), either validated by CT or MRI, were included into this study. They have to be the first time patients of ACI and the disease onset should be shorter than 72 hours, and the cause of the cerebral infarction was cardiac origin, and they were without abnormal renal function, without bleeding history and hemostatic abnormality.

These 70 patients were divided into 2 groups, 35 in control group and the other 35 in observational group. The control group patients were treated with rivaroxaban (anticoagulant), while observational group patients were treated combined rivaroxaban and sodium ozagrel (anti-platelet). Sodium ozagrel inhibits platelet activation and aggregation through specific inhibition of thromboxane (TX) synthetase. Due to the inhibitory effect of ozagrel on platelet activation, platelets cease to become the main lipid surface of thrombin generation.

Beside standard treatment such as enriched oxygen supply, etc, all 70 patients were treated with aspirin 100 mg/tablet/day. Additionally, the control and observational groups were given rivaroxaban 15 mg/tablet/day. On top of rivaroxaban, the observational group received intravenously and daily 80 mg of ozagrel sodium in 500 mL glucose (5%) solution. Everyone was treated for 2 weeks. Both groups have very similar age mean and range (40-70 years).

After 2 weeks of treatment, clinical efficacy (neurological assessments), coagulation lab tests (only CCT is shown) and GCS scores of the two groups were compared. Neurological ssessment was made according to NIH stroke scale (NIHSS). When NIHSS score reduces ≥90%, the disability classification is grade 0, and the treatment is highly efficacious. When NIHSS score reduces to between 46% and 89%, the disability classification is grade 1 to 3, the treatment is considered efficacious. The treatment is no efficacious, when the score is below 46%. The neurological evaluation results are summarized in Table 3.

TABLE 3
Neurological assessment based on NIHSS. The difference between
these 2 groups is statistically significant (P = 0.026),
meaning the patients of observational group generally were
performing better in neurological assessment.

| | | highly | | | non- | | total |
|---|---|---|---|---|---|---|---|
| group | n | efficacious | efficacious | | efficacious | | efficacious |
| observational | 35 | 23 | 10 | (28.6%) | 2 | (5.7%) | 33 (94.3%) |
| | | (65.7%) | | | | | |
| control | 35 | 16 | 8 | (22.9%) | 11 | (31.4%) | 24 (68.6%) |
| | | (45.7%) | | | | | |

The coma assessment was carried out before and after treatment on all subjects, based on Glasgow coma score (GCS) scoring system, which has 15 as full score. The degree of coma is lighter and is reflected with higher score. The recovery based on GCS is summarized in Table 4, together with CCT results.

TABLE 4
The CCT values and GCS scores of different treatment groups
at 2 different time points, before and after treatments.
Both groups started with similar GCS scores, after treatment
both groups showed significant GCS score improvement
(P < 0.05). The GCS scores at T1 are significantly
different between observational and control group (P < 0.01).

| | | Fibrin(ogen) | GCS |
|---|---|---|---|
| group | Time | CCT | score |
| Observational | T0 | 3.97 ± 1.54 | 6.74 ± 1.15 |
| (Rivaroxaban + | T1 | 2.26 ± 1.07 | 11.76 ± 2.88 |
| ozagrel) | difference | −1.71 | 5.02 |
| | in mean | | |
| Control | T0 | 3.93 ± 1.49 | 6.8 ± 1.11 |
| (Rivaroxaban) | T1 | 3.15 ± 1.12 | 9.07 ± 1.92 |
| | difference | −0.78 | 2.27 |
| | in mean | | |

CCT values were significantly reduced after the two different treatments. The observational group has significant reduction compare to the control group, with P value <0.01. The degree of reduction in CCT values strongly correlates with the method of treatments. The more inhibition in the coagulation system, as exemplified by combined anticoagulant (rivaroxaban) and anti-platelet (ozagrel), the better in controlling hyperclottability and hence the better functional recovery of the patients after stroke, even though both treatment methods improved patients outcomes. The general large reduction of CCT values signify the reduction of in vivo thrombin generation process activated at the period around pre- and post-stroke. As the in vivo thrombin generation is inhibited due to the influences of drug treatments, less pro-clotting fibrin-derivatives are generated and more conversion of pro-clotting fibrin-derivatives to anti-clotting fibrin-derivatives through the fibrinolysis system. These anti-clotting fibrin-derivatives, as exemplified by early FDPs, exert their anti-clotting activities on all clot-based assays, such as CCT, and others like PT, aPTT and TT. Indeed, the changes in CCT values are reflected in this study, and so are all these clot-based assays; they are significantly prolonged after the treatments due to the anti-clotting activities of these anti-coagulable factors. The prolongation of these clot-based assays correlates well with the treatment type, with observational group has the more prolonged average results compared to control group (P<0.01).

The two above clinical studies performed in healthy volunteers and stroke patients, were carried out to show that the novel biomarker CCT correlates to in vivo thrombin generation, and its application to monitor clottability change due to hyperclottability diseases and pharmacological intervention is valid. CCT value is greatly influenced by the kind and amount of fibrin-derivatives generated by pathological in vivo thrombin generation, which is active in many diseases, such as cardiovascular diseases, stroke, cancer, inflammatory diseases, etc. The clottability which is mainly reflected by CCT is a powerful and novel biomarker that will have significant application in health and value-based digital healthcare.

The study with stroke patients is consistent with the published clinical studies where combined anti-platelet and anticoagulant treatment increased bleeding risk, due to over-suppression of coagulation system.

### Example 6

In this example, a total of 42 patients aged between 35-86 years (mean age 60.5±25.5) whose blood was collected to study hematological parameter changes during VKA-anticoagulant treatment. These patients received VKA-anticoagulant treatment to reduce their thrombosis risk. These patients were recruited to study the changes in the change in VKA-anticoagulant dosage and the effects of such change on coagulation parameters, including CCT values. Before and after the dosage change, blood samples of the patients were collected for coagulation parameters assessment. The changes or adjustments of the dosages were stably maintained in each patient for at least a month before being asked to return to give blood and receive further medical consultation. Based on the change in the individual's PT-INR (before, TO) and PT-INR (after, T1), there were basically 2 groups, “decrease” (n=20, Table 5) and “increase” (n=22, Table 6) groups. Several parameters were measured to gain insights into the activity of their coagulation system, such as CCT, DD and soluble fibrin monomer (FM, an assay indicating the in vivo thrombin generation).

TABLE 5
The coagulation parameters of the group showing “decrease”
in PT-INR after >1 month of new VKA-dosage adjustment. T0
indicates time before VKA-dosage adjustment, and T1 after the
adjustment. All value is expressed as mean standard ± deviation.

| | | | | DD | FM |
|---|---|---|---|---|---|
| Time | PT (s) | INR | CCT | (mg/L) | (mg/L) |
| T0 | 25.2 ± 3.5 | 2.37 ± 0.44 | 2.25 ± 0.58 | 0.30 ± 0.17 | 4.19 ± 1.14 |
| T1 | 21.8 ± 3.2 | 1.87 ± 0.34 | 3.11 ± 0.59 | 0.71 ± 0.30 | 16.9 ± 7.39 |
| Difference in | −3.4 | −0.50 | 0.86 | 0.41 | 12.71 |
| mean (T1 − T0) | | | | | |
| P | <0.01 | <0.01 | <0.01 | <0.01 | <0.01 |

TABLE 6
The coagulation parameters of the group showing “increase”
in PT-INR after >1 month of new VKA-dosage adjustment. T0
indicates time before VKA-dosage adjustment, and T1 after the
adjustment. All value is expressed as mean standard ± deviation.

| | | | | DD | FM |
|---|---|---|---|---|---|
| Time | PT (s) | INR | CCT | (mg/L) | (mg/L) |
| T0 | 22.3 ± 3.3 | 1.97 ± 0.37 | 2.38 ± 0.45 | 0.40 ± 0.37 | 4.87 ± 2.00 |
| T1 | 26.1 ± 2.7 | 2.39 ± 0.32 | 2.35 ± 0.53 | 0.43 ± 0.4 | 4.65 ± 1.42 |
| Difference in | 3.8 | 0.42 | −0.03 | 0.03 | −0.22 |
| mean (T1 − T0) | | | | | |
| P | <0.01 | <0.01 | 0.787 | 0.589 | 0.231 |

TABLE 7
The coagulation parameters of 4 groups of subjects, based on their
PT-INR values. The 3 different PT-INR groups are the same patients
as in Table 5 and 6. Each of them had 2 different data sets obtained
at T0 and T1. Based on the PT-INR, the 84 data sets fall into one
of the 3 groups. Group “healthy” represents 30 healthy volunteers,
with age 40-78 years (mean age 59 ± 19), who had normal physiological
functions and were not under any pharmacological treatment.

| PT-INR | | | DD | FM |
|---|---|---|---|---|
| group | n | CCT | (mg/L) | (mg/L) |
| <1.8 | 24 | 2.83 ± 0.81 | 0.68 ± 0.42 | 14.61 ± 7.3 |
| 1.8-2.5 | 32 | 2.35 ± 0.55 | 0.36 ± 0.24 | 4.78 ± 3.4 |
| 2.5-3.0 | 28 | 2.43 ± 0.51 | 0.38 ± 0.25 | 4.57 ± 2.0 |
| Healthy | 30 | 2.42 ± 0.30 | 0.33 ± 0.13 | 4.60 ± 1.3 |
| F value | | 3.839 | 9.123 | 39.818 |
| P value | | 0.012 | <0.01 | <0.01 |

The data of Table 7 indicates that the lower the PT-INR value, the higher the in vivo thrombin generation, as revealed by CCT, DD and FM.

### Example 7

This is again another example how anticoagulant treatment reduces pathological in vivo thrombin generation with the concomitant reduction of fibrin-derivatives detectable by CCT. This example is a highly prevalent (>10%) progressive chronic disease, which has strong clinical manifestations of immune and coagulation systems. Chronic obstructive pulmonary disease (COPD) is an inflammatory lung disease that affects respiratory function with concomitant high thrombosis risk. In this example, study subjects were the 70 patients diagnosed with COPD and developed acute exacerbation form of COPD (AECOPD). On top of the standard AECOPD treatments e.g. anti-infection, bronchodilation, phlegm elimination, and oxygen treatment, etc, 32 of these AECOPD patients received no additional treatment and the other 38 of them were treated with low molecular weight heparin calcium (LMWH, 4100 IU anti-FXa/injection every 12 hrs, for 10 days).

Treatment efficacy was evaluated based on several parameters: pulmonary function, blood gas analysis and coagulation parameters. All this information were obtained before and after the LMWH anticoagulant treatment, the same for the control group. The pulmonary function was measured with several methods, such as FEV1 (measurement of forced expiratory volume in 1 sec after bronchodilator was inhaled) and FVC (forced vital capacity, and the ratio of FEV/FVC in % as clinical signs of respiratory performance). Blood gas analysis was obtained by measuring oxygen saturation of blood (SO2), partial pressure of oxygen (PO2) and partial pressure of carbon dioxide (PCO2). Several blood coagulation parameters were measured; they are CCT, DD, PT-INR.

As in the previous examples, this study focused on the dynamic part of the clottability biomarker, CCT, which is a strong and responsive biomarker of in vivo thrombin generation. This study included DD as a compliment indicator of thrombin activity, though not as specific and sensitive as CCT.

Before the start of the treatment, patients of treated and untreated groups were not showing significant differences in these 2 clinical indicators, pulmonary functions and blood gas saturation (all P>0.05, Table 8). After the treatments, both groups showed significant improvement in both indicators (P<0.01, Table 8). The anticoagulant treatment further improved significantly the clinical recovery indicators, as compared to the untreated control (P<0.01, Table 8). This strongly indicates the benefit of anticoagulant, which targets the coagulation system, in treating AECOPD. The use of anticoagulant brought about the change in coagulation parameters, as summarized in Table 9.

TABLE 8
The comparative analyses of the pulmonary functions and blood gas of the AECOPD patients,
treated with LMWH anticoagulant that inhibit coagulation factor Xa (FXa) or without anticoagulant
(control). Both treatments produced significant clinical recovery for pulmonary functions
and blood gas saturation (P < 0.01). LMWH treatment promoted further significant clinical
recovery for these 2 areas, when compared with the control treatment (P < 0.01).

| Treatment | | PO₂ (mmHg) | | PCO₂ (mmHg) | | SO₂(%) | | FEV1 (L) | | FEV1/FVC (%) | |
|---|---|---|---|---|---|---|---|---|---|---|---|
| group | n | Before | After | Before | After | Before | After | Before | After | Before | After |
| LMWH | 38 | 71.5 ± | 78.4 ± | 52.2 ± | 41.9 ± | 90.8 ± | 98.6 ± | 1.36 ± | 1.89 ± | 62.3 ± | 78.4 ± |
| | | 4.3 | 3.6 | 6.4 | 5.5 | 4.9 | 1.4 | 0.17 | 0.36 | 8.7 | 11.4 |
| control | 32 | 73.2 ± | 76.3 ± | 50.2 ± | 45.7 ± | 92.3 ± | 95.7 ± | 1.34 ± | 1.64 ± | 60.2 ± | 67.8 ± |
| | | 4.5 | 4.2 | 7.7 | 5.0 | 4.5 | 1.9 | 0.16 | 0.26 | 9.2 | 10.5 |

The use of anticoagulant LMWH had a strong impact on the coagulation system, and the effects are clearly visible by the biomarker CCT (Table 9). Prior to treatment, both groups showed no significant difference in coagulation parameters, particularly, CCT and DD (all P>0.05, Table 9). Significant differences were only seen within LMWH-treated group in these parameters, before and after treatment (all P<0.01, Table 9). This is in contrast to the control group, before and after treatment, where all parameters remain non-statistical significant (all P>0.05, Table 9). These coagulation parameters indicate that strong inhibition of coagulation factor FXa inhibited, in general, the coagulation system of these 38 AECOPD patients. Similar to stroke (Example 5), reducing the pathological in vivo thrombin generation through antithrombotics produced better clinical recovery for treated patients. The effects of such inhibition have brought about the reduction of in vivo thrombin generation, and facilitated the fibrinolysis process, which is indicated by the lowering of CCT and the reduction in clotting rate as indicated by the increase in PT and PT-INR. Hence, in this example, a strong correlation between CCT and pathological in vivo thrombin generation is demonstrated. The application of CCT/clottability biomarker in many hypercoagulability diseases is further substantiated. Although the AECOPD standard treatment for AECOPD was effective (Table 8, control treatment), halting the coagulation pathway activation or in vivo thrombin generation, and encouraging anticoagulation pathway activation such as fibrinolysis by the supplementary treatment of LMWH or anticoagulant greatly improve the clinical recovery.

TABLE 9
The comparative analyses of blood coagulation parameters
of AECOPD patients received different treatments.

| Treatment | | CCT | | DD (ug/L) | | PT | | INR | |
|---|---|---|---|---|---|---|---|---|---|
| group | n | Before | After | Before | After | Before | After | Before | After |
| LMWH | 38 | 3.97 ± | 2.16 ± | 482.8 ± | 237.4 ± | 9.6 ± | 13.7 ± | 0.87 ± | 1.36 ± |
| | | 0.84 | 0.51 | 253.6 | 74.9 | 0.67 | 0.74 | 0.05 | 0.12 |
| control | 32 | 3.88 ± | 3.63 ± | 476.5 ± | 389.5 ± | 9.5 ± | 9.7 ± | 0.85 ± | 0.93 ± |
| | | 0.82 | 0.75 | 237.2 | 89.6 | 0.70 | 0.68 | 0.04 | 0.04 |

APTT coagulation test was included in the coagulation test panel, but the usefulness is limited due to the inhibitory effect of LMWH on aPTT assay. Because of the possibility of having compounding effects on aPTT data, particularly those that obtained from LMWH-treated patients, the results of aPTT are not included into Table 9. Briefly, control treatment also did not significant change the aPTT results.

This study, unlike antithrombotic treatments of stroke in Example 5, made a comparison study between standard AECOPD treatment with and without anticoagulant. Additionally, this study with AECOPD patients was carried out without anti-platelet treatment.

Comparison study of different populations containing healthy control, stable COPD control and AECOPD individuals in regards to their CCT and DD levels indicates that AECOPD population succumbed to active disease phase or flare-up is showing significant increase in CCT, indicating activation of immune and coagulation systems

### (Table 8)

These examples are representing populational studies, meaning the studies are design to draw conclusions about human population in general.

### Example 8

The use of coagulants such as coagulation factors are getting more frequent due to the rise of acquired factor deficiency, beside the standard use of coagulants for hemophiliacs. There are also situations when patients undergoing anticoagulant treatment are suffering from bleeding, and coagulants and/or by-passing agent (BPA) are administered to stop the bleeding. These coagulants, such as those that supplement particular deficient coagulation factors like FVIII or agents (BPA) that increase efficiency of blood coagulation such as Hemlibra and NovoSeven, are imposing heighten risk of thrombosis. Although the quality of life for hemophiliacs has improved due to the available treatments, but many still suffer from bleedings and chronic illness caused by frequent bleeding.

Administrating too much or little of these coagulants is the main cause of treatment failure. An individualized dosing strategy of such coagulants is needed to optimize the treatment. The current pseudo-pharmacodynamic (PD) response of all these coagulants is mainly monitored by classical coagulation tests such as PT and aPTT, but they are not suitable for many current and future treatment agents. These in vitro diagnostic tests are only able to provide the pharmacokinetics (PK) of the treatment agents; they fail to provide insights if patients are receiving too much or too little treatment agents or drugs, and they are not sufficient to show the efficacy of the one-size-fit-all-treatment. Furthermore, these tests are not able to give any insight regarding the base line in vivo activity of thrombin. Additionally, most of these assays are performed in the absence of many other whole blood components like cellular and subcellular components. In such condition, the clotting reactions are stimulated artificially to check for clotting efficiency in these assays. The assumption that such in vitro data reflects in vivo situation has failed since blood clotting involves the whole blood, not just the sub-fraction of the blood. Without a good method to assess the treatment efficacy, the most commonly accepted clinical endpoints are scoring for bleedings and other adverse events.

Another issue is the variability in the pharmacokinetics (PK) of BPA-treatment in different individuals. Individuals receiving the same dose of BPA are having different PK and hence some individual will need more frequent BPA administration than others, due to inter-individual differences.

Additional issues with these treatments, including gene therapy, recipients frequently develop inhibitors or antibodies (either neutralizing or non-neutralizing) to these agents or drug, and affect the PKs and PDs, differently in individuals. In the example of congenital hemophilia A (CHA), neutralizing antibodies appear in about 30% of the patients and these antibodies reduce the effectiveness of factor VIII (FVIII) treatment, which is still the gold-standard treatment, and depending on the neutralizing activities, the exogenous FVIII is not able to produce full therapeutic activity along the PK concentration range. The remaining about 70% of patients of CHA without neutralizing antibodies can have non-neutralizing antibodies which can greatly influence the PK and availability of FVIII during FVIII-replacement therapy. Methods of immune tolerance induction (ITI) by administering high doses of FVIII, and commonly including other BPAs such as FVII, rFVIIa and activated prothrombin complex concentrate (aPCC) during ITI, were developed and achieved high effectiveness in overcoming these inhibitors. The use of these BPAs, including Hemlibra and other, was associated with adverse events, due to the increased thrombotic risks, and no test or tool available to monitor this treatment process and adverse effects. The critical monitoring method is needed to know if the patients, from before to after ITI treatment are suffering from inhibitor problem and how severe. During the ITI treatment, monitoring is needed to provide guidance on the treatment regimen individually, so that the bleeding and thrombosis risks are minimized. Monitoring is still needed after the ITI for long-term determination if the patients remain stable after the treatment, since relapse of inhibitors is known. Such monitoring diagnostics is lacking and not sensitive for non-neutralizing antibodies. Patients of CHA with only non-neutralizing inhibitors will have very different PKs due to the shorter half-life of FVIII, compared with CHA patients with negligible inhibitors at specific time. Additionally, it is not certain if these patients will have sufficient protection around their trough level. Hence a revolutionary monitoring test system, on top of the existing available tests, is needed to show efficacy of the FVIII treatment along the whole drug PK, individually.

Since it was demonstrated that the levels of von Willebrand factor (VWF) varied individually and this factor influences the stability of FVIII, the PK of exogenous FVIII has to be determined individually, beside the PD determination at the time of blood sampling. This factor adds additional complications into the availability of FVIII for proper function, on top of factors described above. It will be resource-demanding to perform many different tests and measurement for individual CHA, on top of other hemophiliacs like B- and C-types.

Last but not least, hemophiliacs also suffer from dysregulated fibrinolysis due to low thrombin activatable fibrinolysis inhibitor (TAFI) on fibrin clot. Hence, personalized treatment relying on PK or PK-only based therapeutic range is not sufficient to eradicate bleeding completely.

Since the problem of bleedings in hemophiliacs are not completely overcome by these treatment of coagulants or BPAs, here collectively name as drug, there need an individualized dosing regimen to optimize the treatment and reduce the incidence of bleedings. In case of bleedings, these patients treated with some of these drugs faced thrombotic complications. In order to achieve customized treatment, individuals are needed to have their optimized therapeutic method and range determined at different stages of their life, so that their quality of life could further be improved, and much less hospitalization and medical costs.

The existing methods of determining the pharmacokinetics (PK) of these coagulants or BPAs, such as clot-based assays such as aPTT and chromogenic tests, are widely adopted. This individual PK determination is not sufficient, because this information only reflects the drug concentration, and it cannot inform if this drug treatment at all time point of its PK is sufficient to protect patient from bleeding or induce thrombosis. For example drug concentrations at trough level may have very different efficacies for different hemophiliacs. Additionally, the existing tests for FVIII inhibitors are not standardized and are not sufficient to inform the effects of inhibitors to the drug treatment, particularly those non-neutralizing ones that do not interfere with the tests.

Due to the insufficiency of currently available diagnostics, the current treatment for hemophilia is not optimized yet for all hemophiliacs. Currently, there is no biomarker for monitoring the PD effect of the treatment so that hemophiliac treatment can be optimized individually, before breakthrough bleeding and other bleedings occur. All these pains and sufferings could be avoided when the individually optimized therapeutic method and range of a specific single or mixed treatment could be measured and adjusted accordingly, without waiting until the appearing of a catastrophic event.

The solution to above-mentioned dilemmas is found in this invention where a novel biomarker is combined with computational algorithm, which is capable of calculating the risks of thrombosis and bleeding in the individual patients of hemophiliacs during the course of drug treatment with or without the determination of the individual PKs during different treatment methods.

The activity of coagulation pathway can be indicated by the activity of thrombin being activated via intrinsic and extrinsic pathways, with the participation of non-cellular and cellular components in the blood. This thrombin activity leaves its marking by converting fibrinogen into fibrin and fibrin-derived molecules, depending on the conditions, is able to form small soluble and insoluble polymers of various sizes and complexity. These complex and various fibrin-derived molecules or derivatives are indications of thrombin generation in vivo, and they can be pro-coagulable factors such as soluble fibrin, and anti-coagulable factors (sec other section for explanation). Part of the clottability biomarker, CCT, is a strong indicator of these derivatives, as well as fibrinogen level. The dynamic changes of CCT reflect how the coagulation system is responding to certain inducers or treatments, e.g. drugs like BPA, etc. The inducers can be for example immunogen, pathogens, tear and injuries, and they can activate both immune and coagulation systems. Examples for these inducers are viral infection, tissue abrasion and cardiovascular diseases. By following and calculating the appearance and relative abundance of these fibrin derivatives in relation to fibrinogen, the prediction of the risk of thrombosis and bleeding can calculated. This can be done during drug or ITI treatment. Based on this approach, each hemophiliac will receive customized and optimized treatment, and the health care providers will not rely on gut-feeling.

Hemophiliacs rely on frequent drug treatment to restore their normal coagulation function. The treatment frequency is normally dependent on the theoretical drug PK or actual measured PK of that individual. The PKs can range from 1 week to 1 year, dependent on the kind of drug. Here, an example of a drug treatment monitored with the system described in this patent is illustrated to briefly explain how this combined biomarker and computational system is able to provide better information and decision making to health care provider and patient.

Blood samples are tested before and after drug treatment, here using a standard dose of FVIII (PK of 1 week) as an example. A couple of measurement is performed before the treatment, and many measurements are distributed along the drug PK. Clottability biomarker is computed for each time point. To illustrate how this invention is able to solve the current problem in hemophilia treatment, 3 situations representing 3 different patients, who are without FVIII-inhibitor (S1), with neutralizing FVIII-inhibitor (S2) and with non-neutralizing FVIII-inhibitor (S3), under the same treatment as above. At S1, clottabilities are fluctuating up and down around zero to slightly sub-zero before the treatment. Right after the treatment, the clottabilities increase and fluctuate around the same level for one and two days and slowly fluctuate towards zero. Right after the treatment, the coagulation system of patient S1 is restored. Due to the basal activities of immune and coagulation systems, pro-coagulable fibrin-derivatives are dynamically generated and degenerated, hence the clottability fluctuation. Towards the end of the PK, the drug is slowly consumed or metabolized in S1 and clottability is decreasing and is fluctuating around zero, if there is no activation of the extrinsic pathway. The changes in clottability for S1 are far away from the threshold of thrombosis and bleeding.

Before S2 receive the treatment, the clottability fluctuates similarly around zero as S1. Immediately after the treatment, clottability shots up or increases sharply due to high immune activation-induced pro-coagulable fibrin-derivatives and also the brief restoration of coagulation system. Due to the neutralizing antibodies, the drug is very soon rendered ineffective, and then followed by a big dip in clottability due to the much higher ratio of anti-coagulable factors such as FDPs in relation to fibrinogen. This relatively faster reversal of clottability into the negative zone compare to normal is due to the low level of TAFI on fibrin-derivatives. Due to the accelerated fibrinolysis, these fibrin-derivatives are degraded into completion and the clottability returns and fluctuates flatly around zero and sub-zero from day 3 till theoretical day 7 (for S2), if there is no activation of the extrinsic pathway. Therefore, with such invention, health care provider can easily gain insight into this treatment for S2 and can easily decide on alternative treatments e.g. using Hemlibra or NovoSeven.

The case of S3 is much milder than S2. The non-neutralizing antibodies shorten the PK of such treatment to day 5, although FVIII-drug may still participate in the coagulation pathway. Hence, patient S3 does not have sufficient FVIII in his system from day 5 to day 7, and the clottability fluctuates flatly around zero and sub-zero during this period, if there is no activation of the extrinsic pathway.

All these cases, S1-S3, assume negligible extrinsic pathway activation during the course of populational PK. In reality, this is hardly true. Due to daily physiological and physical activities, triggers or inducers of both intrinsic and extrinsic pathways such as infection, inflammation, tissue injury, etc normally exist in everyone. When such inducers are substantial at the time when FVIII is depleted, thrombin is generated in vivo via extrinsic pathway, and this will produce a state where most pro-coagulable fibrin-derivatives are quickly converted to anti-coagulable fibrin-derivatives. This phenomenon is reflected with clottability curve fluctuating further below zero and is a situation that poses higher risk of bleeding for patients, which is illustrated in examples of Table 10.

TABLE 10
Some cases of hemophiliacs and their coagulation parameters. Case
2 has abnormally low PT due to treatment with NovoSeven (FVII)

| case | Hemophilia | age | TT (s) | PT (s) | APTT (s) | CCT |
|---|---|---|---|---|---|---|
| 1 | A | 20 | 18.6 | 12.5 | 61.2 | 1.98 |
| 2 | A | 56 | 18.8 | 6.8 | 64.8 | 1.86 |
| 3 | B | 49 | 17.3 | 12.3 | 134.9 | 2.25 |
| 4 | A | 57 | n.a. | 12 | 82.4 | 1.66 |
| 5 | A | 21 | 18.8 | 13.5 | 65.7 | 1.96 |
| 6 | A | 7 | 17.1 | 13.4 | 123.2 | 2.29 |

All hemophiliacs listed in Table 10 are severe hemophilia patients. B-type hemophilia is much rarer than A-type hemophilia, and this table is a representative of the hemophilia population in their coagulation parameters. All cases here have normal extrinsic pathway (except case 2, due to FVII-treatment) while their intrinsic pathway exhibits dysfunction, because of the deficiency in functional FVIII and FIX (coagulation factor 9). Case 4, who has hypertension, was hospitalized due to bleeding problem. This single CCT data from this rare genetic disease is below the average TFT value of 2.7 (sec Example 4). Therefore, it is very likely that these patients have clottability curve fluctuating below zero or further below zero, when their needed coagulation factor is depleted. Previous studies have shown that protracted infusion of low-dose thrombin, which simulates continuous activation of coagulation system, led to bleeding phenotype. Hence, this patient (Case 4) with inefficient intrinsic pathway (FVIII level<0.1% and long APTT time), enhanced fibrinolysis and negative clottability are setting the stage for high bleeding risk. The high FDP value (16.33 ug/mL; reference range 0-5 ug/mL) confirms the reason for negative clottability (1.66-2.7=−1).

Therefore, this revolutionary calculation method based on clottability biomarker is offering informative and easy monitoring of drug treatment for patients of all hemophilia types, in addition to thrombosis and bleeding risk estimation. This computational approach mainly based on clottability biomarker and assisted by other measurements such as those listed in Table 10 (for patients with hemophilia, other diseases maybe different), is a novel way of assessing thrombosis and bleeding tendency. This is definitely applicable to antithrombotic treatments, illustrated by above examples and main body text, since antithrombotics like anticoagulants inhibits coagulation pathways (e.g., FXa and FIIa) and when the inhibition is stronger and the clottability remain more negative, this is setting a condition for high bleeding risk.

### Example 9

Most current biomarkers have the characteristics of quantitative variations within individuals and making comparisons with different individuals complicated. For example, biomarker A which is found in person A, is a biomarker of the immune system. Biomarker A is found to be different quantitatively within a day or a week, but person A is perfectly healthy and normal. But for person B, who has a chronic illness with involvement of immune system, the biomarker A is found to present in wide quantitative range at different time; sometime person B has the same level of biomarker A as person A, and sometime not the same. Due to the wide variability in quantity, and the biomarkers are normally classified in binary, e.g., true/false, healthy/sick, etc.

With this mathematical approach to data quantification, a lot of information regarding the immune system is lost. Person A, perfectly healthy and normal, may have some minor immune activity, but this information is lost because the quantitative change is still within a cut-off value that define healthy/sick. Additionally, there is no way to determine the basal levels of biomarker A for person A and B, which are likely very different from each other. Without the basal value, it is impossible to measure or define the biological meaning of the fluctuations of the values for person A and B. General practice is obtain as many as possible people representing person A's condition and measure their biomarker A levels. By obtaining a statistical meaningful average value, this value is assumed basal value for every person, even for person B. This is not a correct assumption and can never be used to estimate the activity of the immune system in every individual.

The mathematical approach to circumvent such problem in biomedical research is found in this example. There are at least 2 methods to overcome issues with existing biomarkers. The primary goal here is to obtain the information about coagulation and/or immune systems, mainly about when and how much are they active and inactive. These 2 methods represent 2 different ways to indicate the activity of the systems. When coagulation system is activated, the key enzyme being produce is thrombin. Thrombin converts fibrinogen to fibrin-derivatives which participate to form fibrin-clot. Thrombin also activates many cells including many immune cells, endothelial cell, platelet, etc. Also, when immune system is activated, immune cells and platelet are activating coagulation pathway and more thrombin is produced. CCT is a quantitative expression of fibrinogen and fibrin-derivatives, while TFT fibrinogen only. The first method is the subtracting of CCT with TFT, and this resulted value (clottability) represents the quantitative representation of fibrin-derivatives, which can be pro- and anti-coagulable in nature. If the majority of fibrin-derivatives is pro-coagulable, this signify recent thrombin generation and most fresh pro-coagulable fibrin-derivatives have not been fibrinolytically processed. A few hours later, if the thrombin is not further produced or maintained, most of these fibrin-derivatives become anti-coagulable a short while and become neutral. When multiple clottabilities are available for an individual and is plotted against time, it is a simple visualization of immune and coagulation system over time.

The second method is the CCT/TFT-1. This value represents the ratio between fibrin-derivatives and fibrinogen. These 2 exemplary calculations solve the issues of basal value, intra- and inter-individual variations in translating biomarker into clinical and biological meaning.

The clottability biomarker can be combined with other existing markers (and other clinical classifications, e.g. heart diseases or high blood pressure or diabetes, which are included or input into the Module 1) to provide much improved performance in diagnosis, prognosis and monitoring. These parameters can be combined to enhance machine learning or AI.

The numerical representation of clottability biomarker, either obtained from these two exemplary arithmetic calculations, is a strong indication of in vivo thrombin generation and fibrinolysis. In another word, the larger the value of clottability biomarker, the more in vivo thrombin is generated. Normally, thrombin is generated transiently in vivo when there is either physical injury or immune activation. But in certain conditions such as obesity, diabetes, etc, thrombin is chronically generated and this is reflected in longitudinal measurement of clottability. The higher the value and the longer the value maintained at high value, the poorer the health condition. The rating of health status and risk of thrombosis is based on statistical model reflecting the appropriate population. As an example, when the activity of in vivo thrombin generation is chronically pathogenically high for an individual, the algorithm will match this data to the appropriate representative population with known risk of thrombosis. The health status is reflected and rated on how far away this individual is from the risk of thrombosis and the amplitude and frequency of the clottability (FIGS. 1 and 3). The activation and process of fibrinolysis is reflected in the clottability data, and is an important parameter to be modeled into the risk of thrombosis; the more active fibrinolysis is initiated after increase in clottability, the less risk in thrombosis. Example of bleeding risk calculation is given in Example 8.

The invention further relates to the following embodiments:

1. A method for modelling clottability of a blood sample, the method comprising the steps of
(a) determining clottability of blood samples or providing blood samples with known clottability;
(b) determining and attributing a health status and risk factor for thrombosis and/or bleeding to the donor having provided the respective sample;
(c) determining the pharmacodynamic effect of one or more anticoagulant(s) and/or multiple-drug administrations that contain anticoagulation treatment and the corresponding therapeutic range on the blood sample to reduce the risk determined in (b); and
(d) modelling the clottability of a blood sample after administering the most effective anticoagulant determined in (c) to said blood sample.

2. The method of embodiment 1, wherein determining clottability comprises a combination of a clot-based fibrinogen test and an enzyme-based fibrinogen test.

3. The method of embodiment 2, wherein the enzyme-based fibrinogen test is a clot-independent enzyme-based fibrinogen test.

4. The method of embodiment 2, wherein the clot-based fibrinogen test is selected from determination of the prothrombin time (PT), determination of partial thromboplastin time (PTT), and Clauss-test.

5. The method of any one of embodiments 2 to 4, wherein the enzyme-based fibrinogen test involves catalytic cleavage by a serine endopeptidase.

6. The method of any one of embodiments 2 to 5, wherein the enzyme-based fibrinogen test involves catalytic cleavage of fibrinogen by snake venom serine endopeptidase, preferably by venombin A.

7. The method of any one of embodiments 2 to 6, comprising measuring the proteolytic activity of a serine endopeptidase which is inversely proportional to the fibrinogen level in said sample.

8. A method for providing a personalized anticoagulant treatment regimen of a patient, the method comprising
(a) determining clottability of a blood sample of said patient or providing a known clottability value for said patient;
(b) providing a personalized anticoagulant treatment regimen based on the model of clottability obtained by the method of embodiment 1.

9. The method of embodiment 8, wherein if an ongoing anticoagulant treatment of said patient is not sufficient to reduce the attributed risk factor for thrombosis and/or bleeding, the most effective anticoagulant of embodiment 1 is used for further treatment.

10. The method of embodiment 8, wherein if an ongoing anticoagulant treatment of said patient is reducing the clottability modelled in embodiment 1 below a pre-defined threshold, the most effective anticoagulant of embodiment 1 is used for further treatment.

11. The method of any one of embodiments 1 to 10, wherein the anticoagulant is selected from the group consisting of Vitamin K antagonists, in particular fluindione, warfarin or coumarins, direct thrombin inhibitors, in particular dabigatran, argatroban or hirudin, and direct FXa inhibitors, in particular rivaroxaban, edoxaban, apixaban, heparin, or heparin-like drugs.

12. A computer system or a software able to provide a personalized anticoagulant treatment regimen based on the model of clottability obtained by the method of embodiment 1 and a clottability value as input data, wherein the computer system provides health status, risk assessments, and/or personalized anticoagulant treatment regimen.

### REFERENCES

Janssen M J, Pun S. Fibrinogen test. WO2019/068940 Mackie J, Lawrie A S, Kitchen S, Gaffney P J, Howarth D, Lowe G D, Martin J, Purdy G, Rigsby P, Rumley A. A performance evaluation of commercial fibrinogen reference preparations and assays for Clauss and PT-derived fibrinogen. Thromb Haemost. 2002 June; 87(6):997-1005. PMID: 12083508.
Mackie I J, Kitchen S, Machin S J, Lowe G D; Haemostasis and Thrombosis Task Force of the British Committee for Standards in Haematology. Guidelines on fibrinogen assays. Br J Haematol. 2003 May; 121(3):396-404. doi: 10.1046/j. 1365-2141.2003.04256.x. PMID: 12716361.
Undas A. Determination of Fibrinogen and Thrombin Time (TT). Methods Mol Biol. 2017; 1646:105-110. doi: 10.1007/978-1-4939-7196-1_8. PMID: 28804822.
Zirlik A, Bode C. Vitamin K antagonists: relative strengths and weaknesses vs. direct oral anticoagulants for stroke prevention in patients with atrial fibrillation. J Thromb Thrombolysis. 2017 April; 43(3):365-379. doi: 10.1007/si 1239-016-1446-0. PMID: 27896543; PMCID: PMC5337242.
Kajy M, Mathew A, Ramappa P. Treatment failures of direct oral anticoagulants. Am J Ther. 2019 Oct. 9;
Adeboyeje G, Sylwestrzak G, Barron J J, White J, Rosenberg A, Abarca J, et al. Major Bleeding Risk During Anticoagulation with Warfarin, Dabigatran, Apixaban, or Rivaroxaban in Patients with Nonvalvular Atrial Fibrillation. J Manag Care Spec Pharm. 2017 September; 23(9):968-978.
Ordi-Ros J, Seez-Comet L, Pérez-Conesa M, Vidal X, Riera-Mestre A, Castro-Salomó A, et al. Rivaroxaban versus vitamin K antagonist in antiphospholipid syndrome: A randomized noninferiority trial. Ann Intern Med. 2019 Nov. 19; 171(10):685-694.
Harter K, Levine M, Henderson S O. Anticoagulation drug therapy: a review. West J Emerg Med. 2015 Jan. 12; 16(1):11-17.
Hempenius M, Groenwold R H H, Souverein P C, de Boer A, Klungel O H, Gardarsdottir H. Impact of anticoagulant exposure misclassification on the bleeding risk of direct oral anticoagulants. Br J Clin Pharmacol. 2021 Feb. 5; Brown S C, Sheth K N, Falcone G J. Anticoagulation after intracerebral hemorrhage: a perfect clinical scenario for genetics-based precision medicine. Pharmacogenomics. 2020 April; 21(5):307-309.
Hori M, Connolly S J, Zhu J, Liu L S, Lau C-P, Pais P, et al. Dabigatran versus warfarin: effects on ischemic and hemorrhagic strokes and bleeding in Asians and non-Asians with atrial fibrillation. Stroke. 2013 July; 44(7):1891-1896.
Koverech A, Soldati V, Polidori V, Pomes L M, Lionetto L, Capi M, et al. Changing the Approach to Anticoagulant Therapy in Older Patients with Multimorbidity Using a Precision Medicine Approach. Int J Environ Res Public Health. 2018 Aug. 2; 15(8).

1. A method for classifying the risk and/or status of a subject for inflammatory and/or clotting dysregulation events, the method comprising the steps of:
a) determining or retrieving input data, wherein the input data comprises
i) a clottability biomarker, wherein the clottability biomarker is determined in a blood sample of a subject by at least two different assays; and
ii) patient background information;
b) comparing the input data to a risk and/or status reference pattern, wherein the risk and/or status reference pattern is obtained from at least two reference subjects wherein at least one of the reference subjects has previously had an inflammatory and/or clotting dysregulation event; and
c) classifying the risk and/or status of the subject for inflammatory and/or clotting dysregulation events based on the comparison obtained in (b).

2. The method of claim 1, wherein the assays are a combination of a clot-based fibrinogen test and an enzyme-based fibrinogen test.

3. The method of claim 2, wherein the enzyme-based fibrinogen test is a clot-independent enzyme-based fibrinogen test.

4. The method of claim 2, wherein the clot-based fibrinogen test is selected from determination of the prothrombin time (PT), determination of partial thromboplastin time (PTT), and Clauss-test.

5. The method of claim 2, wherein the enzyme-based fibrinogen test involves catalytic cleavage by a serine endopeptidase.

6. The method of claim 2, wherein the enzyme-based fibrinogen test involves catalytic cleavage of fibrinogen by snake venom serine endopeptidase, preferably by venombin A.

7. The method of claim 2, comprising measuring the proteolytic activity of a serine endopeptidase which is inversely proportional to the fibrinogen level in said sample.

8. The method of claim 1, wherein the patient background information comprises or consists of body weight, sex, age and kidney function.

9. The method of claim 1, wherein the risk and/or status reference pattern is a machine learning model obtained by training on a dataset of reference subjects and wherein comparing the input data to a risk and/or status reference pattern comprises inputting the input data in the machine learning model.

10. The method of claim 1, wherein classifying the risk and/or status of the subject for inflammatory and/or clotting dysregulation event is classifying the risk and/or status of a subject for thrombosis and/or bleeding.

11. A method for prediction of the risk and/or status of a subject for inflammatory and/or clotting dysregulation events, the method comprising the steps of:
a) determining a risk and/or status progression indicator by the steps of:
i) classifying the risk and/or status of a subject for inflammatory and/or clotting dysregulation events according to the method of claim 1 during at a first time point; and
ii) determining or retrieving a clottability biomarker of a blood sample of the subject at a second timepoint, wherein the clottability biomarker is determined in the blood sample of the subject by at least two different assays; and
b) comparing the risk and/or status progression indicator to a prediction reference pattern, wherein the prediction reference pattern is obtained from at least two reference subjects wherein at least one of the reference subjects has previously had an inflammatory and/or clotting dysregulation event and wherein the risk and/or status progression(s) of the reference subject(s) is/are known;
c) predicting the risk and/or status of a subject based on the comparison obtained in (b).

12. The method of claim 11, wherein the prediction reference pattern is a machine learning model obtained by training on a dataset of reference subjects and wherein comparing the risk and/or status progression indicator to a prediction reference pattern comprises inputting the input data in the machine learning model.

13. A method for monitoring treatment response of a subject for inflammatory and/or clotting dysregulation events during treatment, the method comprising the steps of:
a) determining a treatment progression indicator by the steps of:
i) classifying the risk and/or status of a subject for inflammatory and/or clotting dysregulation events according to the method of claim 1 at a first timepoint;
ii) determining or retrieving a clottability biomarker of a blood sample of the subject on at least one second timepoint, wherein the clottability biomarker is determined in the blood sample of the subject by at least two different assays; and
iii)1.) an administration timepoint of the anti-inflammatory, anti-platelet, anticoagulation and/or pro-coagulation and/or anticoagulation compound, wherein the administration timepoint is between the first time point and the second timepoint; preferably the administration timepoint and an administered amount of the anti-inflammatory, anti-platelet, anticoagulation and/or pro-coagulation and/or anticoagulation compound; and
2.) a pharmacodynamic response, wherein the pharmacodynamic response is calculated at least based on the clottability biomarker on the first timepoint, the clottability biomarker on the second time point and the administration timepoint, wherein the pharmacodynamic response is calculated at least based on the clottability biomarker on the first timepoint, the clottability biomarker on the second time point, the administration timepoint and the administered amount;
b) comparing the treatment progression indicator to a treatment response reference pattern, wherein the prediction reference pattern is obtained from at least two reference subjects wherein at least one of the reference subjects previously underwent treatment with an anti-inflammatory, anti-platelet, anticoagulation and/or pro-coagulation compound and wherein the treatment response(s) of the reference subject(s) is/are known;
c) monitoring treatment response of a subject based on the comparison obtained in (b).

14. The method of claim 13, wherein the treatment response reference pattern is a machine learning model obtained by training on a dataset of reference subjects and wherein comparing the treatment progression indicator to a treatment response reference pattern comprises inputting the treatment progression indicator in the machine learning model.

15. An anti-inflammatory, anti-platelet, anticoagulation and/or pro-coagulation compound for use in the treatment of a subject classified as being at risk and/or status for inflammatory and/or clotting dysregulation events according to the method of any one of the claims 1 to 10 and/or predicted to develop risk and/or status for inflammatory and/or clotting dysregulation events according to the method of claim 11.

16. A method of treatment for reducing the risk and/or improving the status of an inflammatory and/or clotting dysregulation event in a subject in need, the method comprising the steps of:
a) administering a therapeutically effective amount of a first anti-inflammatory, anti-platelet, anticoagulation and/or pro-coagulation compound; during a monitoring of the treatment response according to the method of claim 13 to a subject in need; and
b) administering a second anti-inflammatory, anti-platelet, anticoagulation and/or pro-coagulation compound to the subject in need if the treatment response to the first anti-inflammatory, anti-platelet, anticoagulation and/or pro-coagulation compound is insufficient according to the method of claim 13 and proceeding therapy with the first anti-inflammatory, anti-platelet, anticoagulation and/or pro-coagulation compound if the treatment response to the first anti-inflammatory, anti-platelet, anticoagulation and/or pro-coagulation compound is sufficient according to the method of any one of the claims 13 to 14 for reducing the risk and/or improving the status of an inflammatory and/or clotting dysregulation event in the subject in need.

17. The method of claim 13, wherein the compound is a compound selected from the group consisting of Vitamin K antagonists, in particular fluindione, warfarin or coumarins, direct thrombin inhibitors, in particular dabigatran, argatroban or hirudin, and direct FXa inhibitors, in particular rivaroxaban, edoxaban, apixaban, heparin, or heparin-like drugs.

18. The method of claim 13, wherein the compound is a compound selected from the group consisting of: non-steroidal anti-inflammatory drugs, corticosteroids, rapamycin, high density lipoproteins, HDL-cholesterol elevating compounds, rho-kinase inhibitors, anti-malarial agents, acetaminophen, glucocorticoids, steroids, beta-agonists, anticholinergic agents, xanthine derivatives, sulphasalazine, penicillamine, anti-angiogenic agents, dapsone, psoralens, anti TNF agents, anti-IL-1 agents and statins.

19. The method of claim 13, wherein the compound is a compound selected from the group consisting of: irreversible cyclooxygenase inhibitors, adenosine diphosphate (ADP) receptor inhibitors, phosphodiesterase inhibitors, protease-activated receptor-1 (PAR-1) antagonists, glycoprotein IIB/IIIA inhibitors, adenosine reuptake inhibitors, dipyridamole, thromboxane inhibitors and thromboxane receptor antagonists.

20. The method of claim 13, wherein the compound is a coagulation factor that promotes clotting and/or reduces bleeding, preferably a compound selected from the group consisting of FVIII concentrate, Alphanate, Humate-P, NovoSeven, Eloctate, Feiba, prothrombin complex, Hemlibra, and tranexamic acid.

21. A storage device comprising computer-readable program instructions to execute the method according to claim 1.

22. A server comprising the storage device of claim 21, at least one processing device for executing the computer-readable program instructions, and a network connection for receiving the input data.

23. A system for classification, prediction and/or monitoring a treatment response, the system comprising:
a) a measurement setup comprising a container for receiving a blood sample and reagents for determining a clottability biomarker, wherein the clottability biomarker is determined in the blood sample by at least two different assays;
b) a processing device for executing the computer-readable program instructions comprising the storage device of claim 21; and
c) an input and/or retrieval possibility, wherein the input and/or retrieval possibility enables the server and/or the processing device access to the patient background information.
